(19) United States
(12) Patent Application Publication
(10) Pub. No.: US 2007/0113356 A1
(43) Pub. Date: May 24, 2007

(54) Reactive polysaccharide derivatives, their preparation and their use

(75) Inventors: Veronique Hall-Goulle, Dornach (CH); Athanassios Tzikas, Prattein (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(73) Assignee: CIBA SPECIALTY CHEMICALS CORP., TARRYTOWN, NY (US)

(21) Appl. No.: 10/583,012

(22) PCT Filed: Dec. 8, 2004
(86) PCT No.: PCT/EP04/53332

§ 371 Date: Jun. 15, 2006

(30) Foreign Application Priority Data
Dec. 18, 2003 (EP) 03104773.1

(51) Int. Cl. D06M 13/322; C07H 17/02

(52) U.S. Cl. 8/183; 536/18.7; 536/17.4

(57) ABSTRACT

A reactive polysaccharide derivative of formula (1a) or (1b), in which A is —O—, —S— or (1c), Q₁ is hydrogen, the radical —B—A—Z, C₁-C₁₀aryl which is unsubstituted or substituted, C₁-C₁₂alkyl which may be interrupted by oxygen and is unsubstituted or substituted, Q₂ and Q₃ are each independently of the other hydrogen, C₁-C₁₂aryl which is unsubstituted or substituted, C₁-C₁₂alkyl which may be interrupted by oxygen and is unsubstituted or substituted, B is an aliphatic or aromatic bridge member, Z₁ and Z₂ are each independently of the other a reactive radical of the vinylsulfonyl series, the haloacryloyl series or the heterocyclic series, PS is a polysaccharide radical, m is 0, 1 or an integer greater than 1, n is 1 or an integer greater than 1, and the sum of n+m corresponds to the original number of hydroxy groups in the polysaccharide molecule, is useful as a finishing agent for textile fibers and for other applications.

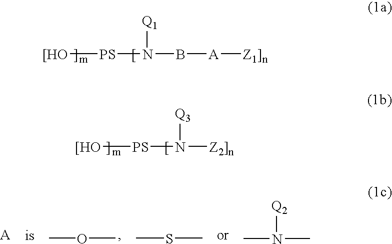

The present invention relates to reactive polysaccharide derivatives, their preparation and their use.

Among the polysaccharide derivatives, which come into consideration for the present invention, cyclodextrins or cydodextrin derivatives may be mentioned in particular.

Due to their particular properties cyclodextrins and cyclodextrin derivatives are used nowadays in numerous technical applications, for example, in the food, pharmaceutical, cosmetic or the chemical industry.

Cyclodextrins are cage like molecules of a cyclic configuration made up of a varying number of D-glucopyranosyl units, such as 6, 7 or 8 units (α-, β- or γ-cyclodextrins), connected by alpha-(1,4)-glycosidic linkages, thereby defining a central cavity. The chemical formula of α-cyclodextrin is depicted below.

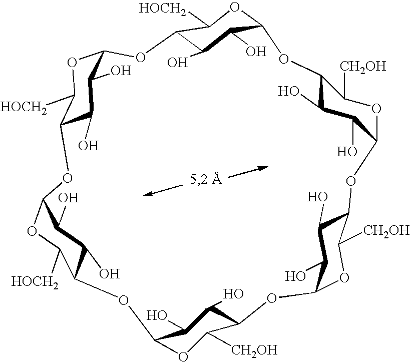

The natural cyclodextrins are produced from starch by the action of cyclodextrin glycosyltransferase (CGTase), an enzyme produced by several organisms, Bacillus macerans being the earliest source. The most stable three dimensional molecular configuration for these cyclic oligosaccharides takes the form of a toroid with the upper (larger) and lower (smaller) opening of the toroid presenting secondary and primary hydroxyl groups, respectively, to the solvent environment. The interior of the toroid is hydrophobic as a result of the electron rich environment provided in large part by the glycosidic oxygen atoms. It is the interplay of atomic (Van der Waals), thermodynamic (hydrogen bonding), and solvent (hydrophobic) forces that accounts for stable complexes that may be formed with chemical substances while in the apolar environment of the cydodextrin cavity. The complex exists in an equilibrium dependent upon the concentrations of the cyclodextrin, the guest molecule and water. The rate at which the associated complex is formed is determined in large part by the accessibility of the guest molecule to the cyclodextrin cavity and the magnitude of the net thermodynamic driving force.

Beside the natural cyclodextrins numerous cyclodextrin derivatives are already known. These derivatives are obtained by conversion with compounds capable of reacting with the hydroxy groups. Examples are the alkylated or hydroxyalkylated derivatives, such as the methylated, hydroxyethylated or hydroxypropylated cyclodextrins, which are accessible by reaction of cyclodextrin with an alkylating agent, such as dimethyl sulfate, ethylene oxide or propylene oxide. Reaction of cyclodextrin with carbonic acid anhydrides or carbonic acid halogenides, such as acetic acid anhydride, benzoyl chloride or naphthoyl chloride, yields the O-acylated derivatives. The respective carboxyl-modified derivatives are obtained, if cydodextrin is reacted with dicarbonic acid anhydrides, such as malonic acid anhydride or succinic acid anhydride. Preparation of these derivatives is known and described in the corresponding prior art.

Due to the number of primary and secondary hydroxyl groups lining the lower and upper ridges of the toroid in the cyclodextrin molecule susceptible to such reactions highly complex mixtures of various isomeric forms of variously substituted cyclodextrin derivatives are obtained. There are, for example, 2²¹−1 or 2,097,151 possible geometric isomers for hydroxypropyl-β-cyclodextrin. The aggregate substitution that takes place is described by a term called the degree of substitution (DS), for example, a hydroxypropyl-β-cyclodextrin with a DS of 5.0 would be composed of a distribution of isomers in which the average number of hydroxypropyl groups per hydroxypropyl-β-cyclodextrin molecule is five. Degree of substitution is usually determined by mass spectrometry (MS) or nuclear magnetic resonance (NMR) spectroscopy and does not give information as to the exact location of the substituents or the distribution of those substituents around the cyclodextrin molecule.

Furthermore, reactive cyclodextrin derivatives comprising at least one nitrogen-containing heterocycle having an electrophilic center are described, for example, in U.S. Pat. No. 5,728,823. Reactive cydodextrin derivatives are able to react with the nucleophilic sites of different substrates and permanently modify the properties of these substrates. U.S. Pat. No. 5,728,823 teaches the preparation of cydodextrins, wherein the reactive heterocycle, such as chlorotriazine, is attached to the cyclodextrin nucleus via an ether bond. These derivatives are prone to hydrolysis, thus limiting their storage stability, which is a disadvantage with regard to their application.

A method for producing reactive cyclodextrins, wherein the reactive group is decoupled from the cyclodextrin nucleus by a flexible spacer, is described in the WO 03/042 449. In this case cyclodextrin is etherified with a bifunctional alkylen compound, such as a halogen-alkylen-amino compound. The terminal functions, e.g. amino groups, thus introduced into the cyclodextrin molecule serve as a nucleophilic site to bind the reactive group precursor.

The methods for preparation according to the prior art are not very selective and yield complex mixtures of multi-substituted derivatives along with the isomers.

Recently, reactive cyclodextrins have been applied also in the textile industry. Accordingly, uncomplexed cydodextrin derivatives are used as finishing agents for the treatment of fiber materials in order to reduce or prevent malodors due to perspiration. The cydodextrin molecules are attached to the fiber material and make sure that the effect stays permanent. Moreover, uncomplexed cydodextrin derivatives allow for the complexation of fragrances and perfumes or antimicrobial substances which are released slowly and impart long-lasting fragrance or a prolonged antimicrobial effect to the finished textile material. Such applications are disclosed, for example, in German Patent No. 40 35 378 and in the WO 02/022 941. The prolonged presence of antimicrobials makes the substrates more hygienic, less prone to cross contamination and fresher.

Nowadays there is an increasing demand for improved reactive polysaccharide derivatives which are useful in various applications, for example, as finishing agents in the textile industry, and which do not show the disadvantage of the prior art systems. In particular they should be stable when stored for a prolonged period of time without hydrolyzation of the reactive moiety. Furthermore, it is desired, e.g. from an application technology point of view, that the derivatives are well defined and do not constitute complicated mixtures of variously substituted cydodextrin derivatives and their isomers.

Furthermore, improved reactive polysaccharide derivatives are required in the textile industry, which may be applied concomitantly to dyeing or printing without carrying out a separate finishing step.

Accordingly, it is the subject of the present invention to provide a reactive polysaccharide derivative which corresponds to the following formula 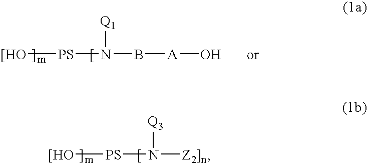
in which
A is —O—, —S— or 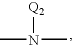
Q₁ is hydrogen, the radical —B—A—Z₁, C₁-C₁₀aryl which is unsubstituted or substituted,
C₁-C₁₂alkyl which may be interrupted by oxygen and is unsubstituted or substituted,
Q₂ and Q₃ are each independently of the other hydrogen, C₁-C₁₀aryl which is unsubstituted or substituted, C₁-C₁₂alkyl which may be interrupted by oxygen and is unsubstituted or substituted,
B is an aliphatic or aromatic bridge member,
Z₁ and Z₂ are each independently of the other a reactive radical,
PS is a polysaccharide radical,
m is 0, 1 or an integer greater than 1,
n is 1 or an integer greater than 1, and
   the sum of n+m corresponds to the original number of hydroxy groups in the polysaccharide molecule.

Q₁, Q₂ and Q₃ independently of the other as C₁-C₁₂alkyl which may be interrupted by oxygen are straight-chain or branched and are, for example, methyl, ethyl, n- or isopropyl, n-, iso-, sec- or tert-butyl, n-pentyl, neo-pentyl, n-hexyl, n-heptyl, n-octyl, n-nonyl, n-decyl, n-undecyl or n-dodecyl. The alkyl radicals mentioned may be interrupted by e.g. 1, 2 or 3 oxygen atoms and are unsubstituted or substituted by e.g. C₂-C₄alkanoylamino, such as acetylamino or propionylamino; C₁-C₄alkoxy, such as methoxy or ethoxy; hydroxy; sulfo; sulfato; carboxy; cyano; carbamoyl; sulfamoyl; β-sulfatoethylsulfonyl; β-chloroethylsulfonyl; or C₁-C₁₀aryl, such as phenyl or naphthyl, which in turn is unsubstituted or substituted. Furthermore, amino comes into consideration as a substituent for Q₁ in the meaning of C₁-C₁₂alkyl. Substituents which come into consideration for C₁-C₁₀aryl are e.g. C₂-C₄alkanoylamino, such as acetylamino or propionylamino; C₁-C₄alkyl, such as methyl or ethyl; C₁-C₄alkoxy, such as methoxy or ethoxy; halogen, such as fluorine, chlorine or bromine; hydroxy; sulfo; nitro; carboxy; cyano; carbamoyl or sulfamoyl, preferably methyl; ethyl; methoxy; ethoxy; chlorine or sulfo. Preferred substituents for C₁-C₁₂alkyl are methoxy; ethoxy; hydroxy; sulfo; carboxy; sulfato; or phenyl or naphthyl which in turn are unsubstituted or substituted by methyl, ethyl, methoxy, ethoxy, chlorine or sulfo; in particular phenyl or hydroxy; and especially phenyl.

Examples of the substituted alkyl radicals and the alkyl radicals interrupted by oxygen for Q₁, Q₂ and Q₃ are: β-hydroxyethyl, 2-(β-hydroxyethoxy)ethyl, 2-[2-(β-hydroxyethoxy)ethoxy]-ethyl, 2-(β-chloroethylsulfonyl)ethyl, 2-(β-sulfatoethylsulfonyl)ethyl, β-sulfatoethyl, β-sulfoethyl, carboxymethyl, β-carboxyethyl or benzyl.

Q₁, Q₂ and Q₃ independently of the other as C₁-C₁₀aryl are, for example, phenyl or naphthyl. The aryl radicals mentioned are unsubstituted or substituted by e.g. C₂-C₄alkanoylamino, such as acetylamino or propionylamino; C₁-C₄alkyl, such as methyl or ethyl; C₁-C₄alkoxy, such as methoxy or ethoxy; halogen, such as fluorine, chlorine or bromine; hydroxy; sulfo; nitro; carboxy; cyano; carbamoyl or sulfamoyl, preferably methyl; ethyl; methoxy; ethoxy; chlorine or sulfo.

In a particular embodiment of the present invention one of Q₁ and Q₂ is hydrogen and the other one of Q₁, and Q₂ is C₁-C₁₀aryl which is unsubstituted or substituted or C₁-C₁₂alkyl which may be interrupted by oxygen and is unsubstituted or substituted, whereby the radical Q₁ furthermore may correspond to a radical of formula —B—A—Z₁.

In another particular embodiment of the present invention Q₂ is hydrogen and Q₁ is a radical of formula —B—A—Z₁, C₁-C₁₀aryl which is unsubstituted or substituted or C₁-C₁₂alkyl which may be interrupted by oxygen and is unsubsttuted or substituted.

Q₁, Q₂ and Q₃ in the meaning of C₁-C₁₂alkyl which may be interrupted by oxygen and is unsubstituted or substituted are preferably C₁-C₆alkyl and especially C₁-C₄alkyl, either of which may be interrupted by oxygen and is unsubstituted or substituted as given above. Preferred are the uninterrupted and unsubstituted radicals and the radicals substituted by phenyl. Very important are benzyl and C₁-C₄alkyl, such as methyl or ethyl.

Preferably A is —S— or 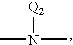
in particular

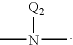

In a preferred embodiment Q₁ is hydrogen, benzyl and C₁-C₄alkyl which is unsubstituted or substituted by amino, or the radical —B—A—Z₁, and Q₂ and Q₃ are each independently of the other hydrogen, benzyl and C₁-C₄alkyl.

In a particularly preferred embodiment Q₁, Q₂ and Q₃ are each independently of the other hydrogen, benzyl or C₁-C₄alkyl.

An aliphatic bridge member B is, for example, a C₂-C₁₂alkylene radical, which is unsubstituted or substituted, e.g. by hydroxy, sulfo, sulfato, cyano or carboxy, and which may be interrupted by 1, 2 or 3 members from the group —N(R₁ₐ)— and —O—, in particular —O—, in which R₁ₐ is hydrogen or C₁-C₄alkyl, such as methyl or ethyl, or R₁ₐ has the meaning and preference as indicated for Z₁ below. Preferred substituents of the alkylene radicals B are hydroxy, sulfo or sulfato, in particular hydroxy or sulfato.

Aliphatic bridge members B are furthermore, for example, C₅-C₉-cycloalkylene radicals, in particular cyclohexylene radicals. The cycloalkylene radicals mentioned are unsubsttuted or substituted, e.g. by C₁-C₄alkyl, C₁-C₄alkoxy, C₂-C₄alkanoylamino, sulfo, halogen or carboxy, in particular by C₁-C₄alkyl.

An aliphatic bridge member B comprises also, for example, methylenecyclohexylene, ethylenecyclohexylene or methylenecyclohexylenemethylene radicals which are unsubstituted or substituted in the cyclohexylene ring, e.g. by C₁-C₄alkyl, in particular methyl.

Moreover, there comes into consideration as an aliphatic bridge member B, for example, C₁-C₄alkylenephenylen-C₁-C₄alkylene, such as methylenephenylenemethylene.

The radical of the formula 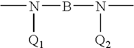
includes also, for example, a radical of the formula 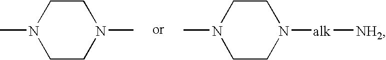
in which alk is C₁-C₄alkylene, such as ethylene.

An aromatic bridge member B is, for example, C₁-C₆alkylphenylene, such as methylenephenylene, phenylene or naphthylene each unsubstituted or substituted in the aryl ring, e.g. by C₁-C₄alkyl, C₁-C₄alkoxy, C₂-C₄alkanoylamino, sulfo, halogen or carboxy, or a radical of the formula 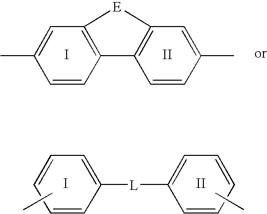
in which the benzene rings I and II are unsubstituted or substituted, e.g. by C₁-C₄alkyl, C₁SO₂—NH— or —SO₂— and L is the direct bond or a C₂-C₁₀alkylene radical, which can be interrupted by 1, 2 or 3 oxygen atoms, or L is a bridge member of the formula —CH═CH—, —N═N—, —NH—, —CO—, —NH—CO—, —NH—SO₂—, —NH—CO—NH—, —O—, —S—, —SO₂— or 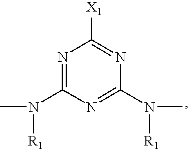
in which X₁ is chloro or fluoro, in particular chloro, and R₁ is hydrogen or C₁-C₄alkyl. An aromatic bridge member B is preferably phenylene, which can be substituted as defined above. Preferably, the aromatic bridge members B are unsubstituted or substituted by sulfo.

B is preferably an aliphatic bridge member.

B is particularly preferably a C₂-C₁₂alkylene radical, especially a C₂-C₆alkylene radical, which is unsubstituted or substituted by hydroxy, sulfo, sulfato, cyano or carboxy, in particular hydroxy or sulfato, and may be interrupted by 1, 2 or 3 members —N(R₁ₐ)— and —O—, in which R₁ₐ is hydrogen or C₁-C₄alkyl, or R₁ₐ has the meaning and preference as given for Z₁ below, and especially is uninterrupted and unsubstituted.

Examples for B as the unsubstituted and uninterrupted C₂-C₆alkylene radicals are 1,2-ethylene, 1,3-propylene, 1,2-propylene, 1,4-butylene, 1,3-butylene, 1,5-pentylene, 3,5-pentylene, 1,6-hexylene, 2,5-hexylene, 4,6-hexylene and the radicals of the formula

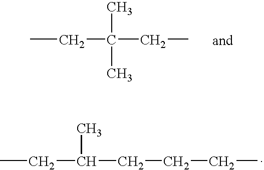

Examples for B as the substituted and/or interrupted C₂-C₆alkylene radicals are 2-hydroxy-1,3-propylene, 2-sulfato-1,3-propylene and the radicals of the formula —(CH₂)₂—O—(CH₂)₂—, —(CH₂)₂—O—(CH₂)₂—O—(CH₂)₂—, —(CH₂)₂—O—(CH₂)₃—, —(CH₂)₂—NH—(CH₂)₂—, —(CH₂)₂—NH—(CH₂)₂—NH—(CH₂)₂— oder —(CH₂)₂—NH—(CH₂)₂—.

Especially important bridge members B are 1,2-ethylene, 1,3-propylene and 1,2-propylene.

Reactive radicals Z₁ and Z₂ are of the vinylsulfonyl series, the haloacryloyl series or the heterocydic series.

Z₁ and Z₂ are capable of reacting with functional groups of a suitable reactant or a suitable substrate, such as a polymer or a textile fiber material, for example, with the hydroxyl groups of cellulose, with the amino, carboxy, hydroxy and thiol groups in wool and silk or with the amino and possibly carboxy groups of synthetic polyamides to form covalent chemical bonds.

Reactive radicals Z₁ and Z₂ from the group of the vinylsulfonyl series comprise, for example, alkylsulfonyl radicals substituted by a removable atom or by a removable group or alkenylsulfonyl radicals which are unsubstituted or substituted by a removable atom or by a removable group. The said alkylsulfonyl and alkenylsulfonyl radicals contain generally from 2 to 8, preferably from 2 to 4, and especially 2, carbon atoms.

Reactive radicals Z₁ and Z₂ from the group of the haloacryloyl series comprise, for example, alkanoyl radicals or alkenoyl radicals substituted by at least one removable halogen atom, such as fluorine, chlorine or bromine. The said alkanoyl and alkenoyl radicals contain generally from 2 to 8, preferably 3 or 4, and especially 3, carbon atoms.

Examples of suitable reactive radicals Z₁ and Z₂ from the group of the heterocyclic series comprise heterocyclic radicals that contain 4-, 5- or 6-membered rings and that are substituted by a removable atom or by a removable group. Suitable heterocyclic radicals are, for example, those that contain at least one removable substituent bonded to a heterocyclic radical, inter alia those that contain at least one reactive substituent bonded to a 5- or 6-membered heterocyclic ring, for example to a monoazine, diazine, pyridine, pyrimidine, pyridazine, pyrazine, thiazine, oxazine or asymmetrical or symmetrical triazine ring, or to such a ring system that has one or more fused-on aromatic rings, for example a quinoline, phthalazine, quinazoline, quinoxaline, acridine, phenazine or phenanthridine ring system.

Radicals Z₁ and Z₂ from the group of the vinylsulfonyl series comprise, for example, a radical —SO₂—CH═CH₂ or SO₂—CH₂—CH₂—U wherein U is a leaving group.

Radicals Z₁ and Z₂ from the group of the haloacryloyl series comprise, for example, a radical —CO—CH(Hal)—CH₂(Hal) or —CO—C(Hal)═CH₂ wherein Hal is chlorine or bromine.

Radicals Z₁ and Z₂ from the group of the heterocyclic series comprise, for example, a halotriazine, halopyrimidine or haloquinoxaline radical, especially a halotriazine radical, wherein the halogen is fluorine or chlorine.

In an interesting embodiment of the present invention Z₂ is a radical of the vinylsulfonyl series or the acryloyl series.

The reactive radical Z₁ and the radical of the formula 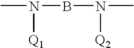
as well as the reactive radical Z₂ and the radical of the formula 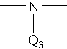
may be connected to one another by way of a bridging member. Suitable bridging members include, besides a direct bond, a very wide variety of radicals. For example, the bridging member is an aliphatic or aromatic radical; the bridging member may also be composed of various such radicals. A suitable aliphatic radical is, for example, an alkylene radical having from 1 to 6 carbon atoms, or a branched isomer thereof. The carbon chain of the alkylene radical may be interrupted by a hetero atom, for example an oxygen atom. A suitable aromatic radical is, for example, a phenyl radical which may be substituted, for example, by C₁-C₄alkyl, such as methyl or ethyl, C₁-C₄alkoxy, such as methoxy or ethoxy, halogen, such as fluorine, bromine or, especially, chlorine, carboxy or by sulfo. In the case of Z₁ the bridging member may contain at least one functional group, for example the carbonyl- or the sulfonyl group.

Such reactive radicals Z₁ and Z₂ are known per se and large numbers of them are described in the art of reactive dyestuffs, for example, in Venkataraman “The Chemistry of Synthetic Dyes” Volume 6, pages 1-209, Academic Press, New York, London 1972, EP-A-625 549 and U.S. Pat. No. 5,684,138.

Preferably a reactive radical Z₁ corresponds to formula (2a), (2b), (2c), (2d) or (2e) 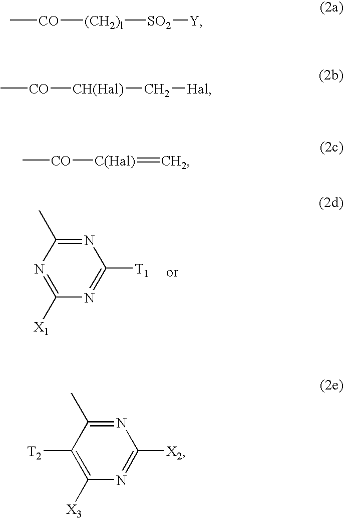
in which
Hal is chlorine or bromine,
X₁ is halogen, pyridinium, 3-carboxypyridin-1-yl or 3-carbamoylpyridin-1-yl, or a reactive radical of formula (3a), (3b), (3c), (3d), (3e) or (3f) 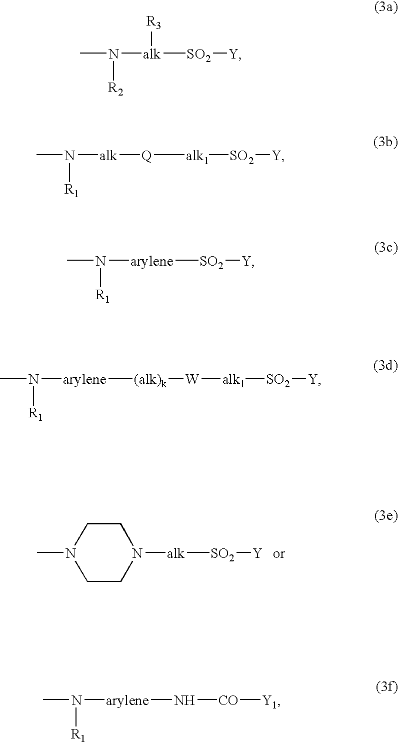
   in which
R₁ is hydrogen or C₁-C₄alkyl,
R₂ is hydrogen, C₁-C₄alkyl unsubstituted or substituted by hydroxy, sulfo, sulfato, carboxy or by cyano, or a radical 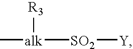
R₃ is hydrogen, hydroxy, sulfo, sulfato, carboxy, cyano, halogen, C₁-C₄alkoxycarbonyl, C₁-C₄alkanoyloxy, carbamoyl or a group —SO₂—Y,
alk and alk₁ are each independently of the other linear or branched C₁-C₆alkylene, arylene is a phenylene or naphthylene radical unsubstituted or substituted by sulfo, carboxy, C₁-C₄alkyl, C₁-C₄alkoxy or by halogen,
Q is a radical —O— or —NR₁— wherein R₁ is as defined above,
W is a group —SO₂—NR₂—, —CONR₂— or —NR₂CO— wherein R₂ is as defined above,
Y is vinyl or a radical —CH₂—CH₂—U and U is a group removable under alkaline conditions,
Y₁ is a group —CH(Hal)—CH₂—Hal or —C(Hal)═CH₂ and Hal is chlorine or bromine, and
I is an integer from 1 to 6 and k is a number 0 or 1, and
X₂ is halogen or C₁-C₄alkylsulfonyl,
X₃ is halogen or C₁-C₄alkyl,
T₁ has independently the same definitions as X₁ above, or is a non-reactive substituent, and
T₂ is hydrogen, cyano or halogen.

Preferably a reactive radical Z₂ corresponds to formula (4a), (4b), (4c), (4d), (4e) or (4f) 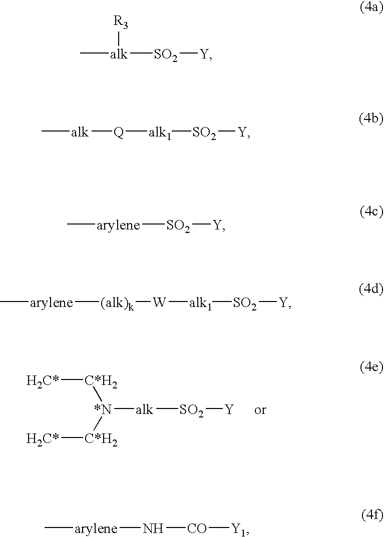
in which
R₃, alk, alk₁, arylene, W, Y, Y₁ and k are as defined above, Q is a radical —O— and the atoms indicated with an asterisk in the reactive radical of formula (4e) together with the radical of formula 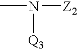
form a piperazine ring.

As a group U removable under alkaline conditions there come into consideration, for example, —Cl, —Br, —F, —OSO₃H, —SSO₃H, —OCO—CH₃, —OPO₃H₂, —OCO—C₆H₅, —OSO₂—C₁-C₄alkyl and —OSO₂—N(C₁-C₄alkyl)₂. U is preferably a group of formula —Cl, —OSO₃H, —SSO₃H, —OCO—CH₃, —OCO—C₆H₅ or —OPO₃H₂, especially —Cl or —OSO₃H and more especially —OSO₃H.

Examples of suitable radicals Y are accordingly vinyl, β-bromo- or β-chloro-ethyl, β-acetoxy-ethyl, β-benzoyloxyethyl, β-phosphatoethyl, β-sulfatoethyl and β-thiosulfatoethyl. Y is preferably vinyl, β-chloroethyl or β-sulfatoethyl, and especially vinyl or β-sulfatoethyl.

R₁ is preferably hydrogen, methyl or ethyl, and especially hydrogen.

R₂ is preferably hydrogen or C₁-C₄alkyl, for example methyl, ethyl, propyl, isopropyl, butyl, isobutyl, sec-butyl and tert-butyl, and especially hydrogen, methyl or ethyl. R₂ is more especially hydrogen.

R₃ is preferably hydrogen.

I is preferably a number 2, 3 or 4, and especially a number 2 or 3.

More especially, I is the number 3.

For a non-reactive substituent T₁ there come into consideration, for example, the following radicals:
hydroxy;
C₁-C₄alkoxy, e.g. methoxy, ethoxy, n- or iso-propoxy, n-, sec-, iso- or tert-butoxy, especially methoxy or ethoxy; the mentioned radicals are unsubstituted or substituted in the alkyl moiety, for example, by C₁-C₄alkoxy, hydroxy, sulfo or by carboxy;
C₁-C₄alkylthio, e.g. methylthio, ethylthio, n- or iso-propylthio or n-butylthio; the mentioned radicals are unsubstituted or substituted in the alkyl moiety, for example, by C₁-C₄alkoxy, hydroxy, sulfo or by carboxy;
amino;
N-mono- or N,N-di-C₁-C₆alkylamino, preferably N-mono- or N,N-di-C₁-C₄alkylamino; the mentioned radicals are unsubstituted or substituted in the alkyl moiety, e.g. by C₂C₄-alkanoylamino, C₁-C₄alkoxy, hydroxy, sulfo, sulfato, carboxy, cyano, carbamoyl or by sulfamoyl and uninterrupted or interrupted in the alkyl moiety by oxygen; examples that may be mentioned include N-methylamino, N-ethylamino, N-propylamino, N,N-dimethylamino and N,N-diethylamino, N-β-hydroxyethylamino, N,N-di-β-hydroxyethylamino, N-2-(β-hydroxyethoxy)ethylamino, N-2-[2-(β-hydroxyethoxy)ethoxy]ethylamino, N-β-sulfatoethylamino, N-β-sulfoethylamino, N-carboxymethylamino, N-β-carboxy-ethylamino, N-α,β-dicarboxy-ethylamino, N-α,γ-dicarboxypropylamino and N-ethyl-N-β-hydroxyethylamino or N-methyl-N-β-hydroxyethylamino;
C₅-C₇cycdoalkylamino, for example, cyclohexylamino, which includes both the unsubstituted radicals and the radicals substituted in the cydoalkyl ring, e.g. by C₁-C₄alkyl, especially methyl, or by carboxy;
phenylamino or N—C₁-C₄alkyl-N-phenylamino, which includes both the unsubstituted radicals and the radicals substituted in the phenyl ring, e.g. by C₁-C₄alkyl, C₁-C₄alkoxy, C₂-C₄-alkanoylamino, carboxy, carbamoyl, sulfo or by halogen, such as 2-, 3- or 4-chlorophenylamino, 2-, 3- or 4-methylphenylamino, 2-, 3- or 4-methoxyphenylamino, 2-, 3- or 4-sulfophenylamino, disulfophenylamino or 2-, 3- or 4-carboxyphenyl-amino;
naphthylamino unsubstituted or substituted in the naphthyl ring, e.g. by sulfo, preferably the radicals substituted by from 1 to 3 sulfo groups, such as 1- or 2-naphthylamino, 1-sulfo-2-naphthylamino, 1,5-disulfo-2-naphthylamino or 4,8-disulfo-2-naphthylamino;
benzylamino unsubstituted or substituted in the phenyl moiety, e.g. by C₁-C₄alkyl, C₁-C₄-alkoxy, carboxy, sulfo or by halogen; or
piperidino or morpholino.

As a non-reactive radical, T₁ is preferably C₁-C₄alkoxy, C₁-C₄alkylthio, hydroxy, amino, N-mono- or N,N-di-C₁-C₄alkylamino unsubstituted or substituted in the alkyl moiety by hydroxy, sulfato or by sulfo, morpholino, or phenylamino or N—C₁-C₄alkyl-N-phenylamino (wherein the alkyl is unsubstituted or substituted by hydroxy, sulfo or by sulfato) each unsubstituted or substituted in the phenyl ring by sulfo, carboxy, acetylamino, chlorine, methyl or by methoxy, or naphthylamino unsubstituted or substituted by from 1 to 3 sulfo groups.

Especially preferred non-reactive radicals T₁ are amino, N-methylamino, N-ethylamino, N-β-hydroxyethylamino, N-methyl-N-β-hydroxyethylamino, N-athyl-N-β-hydroxyethylamino, N,N-di-β-hydroxyethylamino, morpholino, 2-, 3- or 4-carboxyphenylamino, 2-, 3- or 4-sulfo-phenylamino or N—C₁-C₄alkyl-N-phenylamino.
X₁ is preferably halogen, such as fluorine, chlorine or bromine, especially chlorine or fluorine and in particular chlorine.
T₂, X₂ and X₃ as halogen are, for example, fluorine, chlorine or bromine, especially chlorine or fluorine.
X₂ as C₁-C₄alkylsulfonyl is, for example, ethylsulfonyl or methylsulfonyl and especially methylsulfonyl.
X₃ as C₁-C₄alkyl is, for example, methyl, ethyl, n- or iso-propyl, n-, iso- or tert-butyl and especially methyl.
X₂ and X₃ are preferably each independently of the other chlorine or fluorine.
T₂ is preferably cyano or chlorine.
Hal is preferably bromine.
alk and alk₁ are each independently of the other, for example, a C₁-C₆alkylene radical, such as a methylene, ethylene, 1,3-propylene, 1,4-butylene, 1,5-pentylene or 1,6-hexylene radical or a branched isomer thereof.
alk and alk₁ are preferably each independently of the other a C₁-C₄alkylene radical and especially an ethylene radical or propylene radical. arylene is preferably a 1,3- or 1,4-phenylene radical unsubstituted or substituted, for example, by sulfo, methyl, methoxy or by carboxy, and especially an unsubstituted 1,3- or 1,4-phenylene radical.
Q is preferably —NH— or —O— and especially —O—.
W is preferably a group of formula —CONH— or —NHCO—, especially a group of formula —CONH—.
k is preferably the number 0.

The reactive radicals of formulae (3a) to (3f) are preferably those wherein W is a group of formula —CONH—, R₁ is hydrogen, methyl or ethyl, R₂ and R₃ are each hydrogen, Q is a radical —O— or —NH—, alk and alk₁ are each independently of the other ethylene or propylene, arylene is phenylene unsubstituted or substituted by methyl, methoxy, carboxy or by sulfo, Y is vinyl, β-chloroethyl or β-sulfatoethyl, Y₁ is —CHBr—CH₂Br or —CBr═CH₂ and k is the number 0.

The reactive radicals of formulae (4a) to (4f) are preferably those wherein W is a group of formula —CONH—, R₃ is hydrogen, Q is a radical —O—, alk and alk₁ are each independently of the other ethylene or propylene, arylene is phenylene unsubstituted or substituted by methyl, methoxy, carboxy or by sulfo, Y is vinyl, β-chloroethyl or β-sulfatoethyl, Y₁ is —CHBr—CH₂Br or —CBr═CH₂ and k is the number 0.

More preferably Z₁ is a radical of formula (2a), (2b), (2c) or (2d) as defined above, in which Y is vinyl, β-chloroethyl or β-sulfatoethyl, Hal is bromine, I is a number 2 or 3, X₁ is halogen, T₁ is C₁-C₄alkoxy, C₁-C₄alkylthio, hydroxy, amino, N-mono- or N,N-di-C₁-C₄alkylamino unsubstituted or substituted in the alkyl moiety by hydroxy, sulfato or by sulfo, morpholino, or phenylamino or N—C₁-C₄alkyl-N-phenylamino each unsubstituted or substituted in the phenyl ring by sulfo, carboxy, acetylamino, chlorine, methyl or by methoxy and wherein the alkyl is unsubstituted or substituted by hydroxy, sulfo or by sulfato, or naphthylamino unsubstituted or substituted by from 1 to 3 sulfo groups, or is a fibre-reactive radical of formula (3a′), (3b′), (3c′), (3d′) or (3f′) 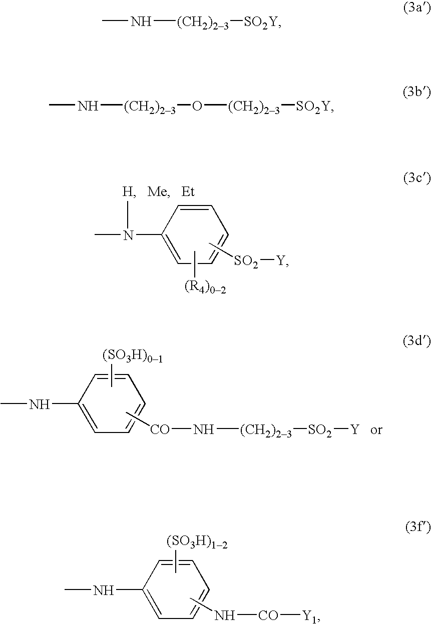
especially (3c′) or (3d′) and in particular (3c′), in which
(R₄)₀₋₂ is 0 to 2 identical or different substituents from the group of methyl, methoxy and sulfo, especially methyl and methoxy,
Y is as defined above, and
Y₁ is a group —CH(Br)—CH₂—Br or —C(Br)═CH₂.

In the radical of formula (3c′) Me is methyl and Et is ethyl. Beside hydrogen, the said radicals come into consideration as the substituent attached to the nitrogen atom.

More preferably Z₂ is a radical of formula (4a′), (4b′), (4c′), (4c′), (4d′) or (4f′) 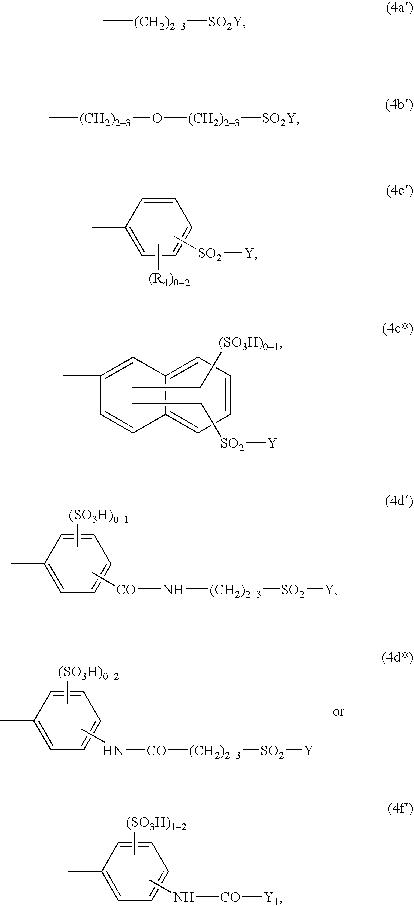
especially (4c′), (4c*), (4d′) or (4d*) and in particular (4c′), in which
(R₄)₀₋₂ is 0 to 2 identical or different substituents from the group of methyl, methoxy and sulfo, especially methyl and methoxy,
Y is vinyl, β-chloroethyl or β-sulfatoethyl, and
Y₁ is a group —CH(Br)—CH₂—Br or —C(Br)═CH₂.

The radical of formula (3c′) is preferably a radical of formula 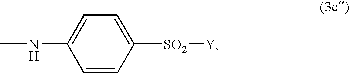
wherein Y has the meanings and preferred meanings given above.

The radical of formula (4c′) is preferably a radical of formula 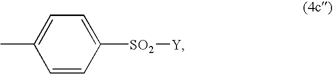
wherein Y has the meanings and preferred meanings given above.

In an important embodiment the reactive radical Z₁ corresponds to a radical of formula (2a) or (2d), especially (2d), in which X₁, T₁, Y and I are as defined and preferred above.

Sulfo groups present in the reactive polysaccharide derivatives of formulae (1a) and (1b) are each either in the form of their free acid or preferably in the form of a salt thereof. Salts that come into consideration include, for example, the alkali metal, alkaline earth metal or ammonium salts, salts of an organic amine or mixtures thereof. Examples that may be mentioned are sodium, lithium, potassium and ammonium salts, the salt of mono-, di- or tri- ethanolamine or Na/Li or Na/Li/NH₄ mixed salts.

The present invention relates also to a process for the preparation of the reactive polysaccharide derivatives of formula (1a) or (1b), which process comprises the steps of
(i) introducing at least one leaving group into the polysaccharide molecule by reaction of a polysaccharide compound of the formula 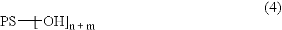
   with at least n molar equivalents of a leaving group precursor P* to yield the compound of formula 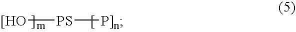
(ii) reacting the compound of formula (5) with at least n molar equivalents of the compound of the formula 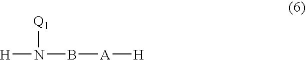
   to yield the compound of formula 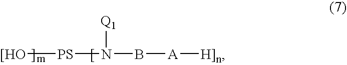
   and allowing the compound of the formula (7) to react with at least n molar equivalents of the compound of the formula
   Z₁—X (8),
   or reacting the compound of formula (5) with at least n molar equivalents of the compound of the formula 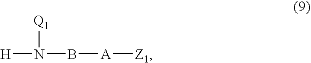
   or reacting the compound of formula (5) with at least n molar equivalents of the compound of the formula 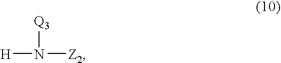
   wherein
   Q₁, Q₃, A, B, Z₁, Z₂, m and n are as defined and preferred above, and X and P are a leaving group, and PS is as defined hereinafter.

The sum of n+m corresponds to the original number of hydroxy groups in the reactive polysaccharide derivative of formula (1a) or (1b), i.e. the total number of hydroxy groups in the polysaccharide compound of formula (4), which is, for example, 18 for α-cyclodextrin, 21 for β-cyclodextrin and 24 for γ-cyclodextrin. n corresponds to the number of the radicals of the formula 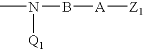
in the polysaccharide derivative of the formula (1a) or to the number of the radicals of the formula 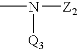
in the polysaccharide derivative of formula (1b).

n is at least 1 and does not correspond to the average number of the radicals of the formula 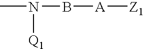
in the polysaccharide derivative of the formula (1a) or to the average number of the radicals of the formula 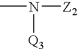
in the polysaccharide derivative of formula (1b), i.e. the average degree of substitution (DS), which refers to a population of polysaccharide derivatives. The average degree of substitution (DS) can also be a noninteger, for example, a number smaller than 1, such as 0.3.

The upper limit of n is determined by the total number n+m of hydroxy groups in the polysaccharide compound of formula (4) available for the substitution reaction according to the preparation process given above. Theoretically, the maximum degree of substitution, i.e. upper limit of n, is 18 for α-cyclodextrin, 21 for β-cyclodextrin, and 24 for γ-cyclodextrin. In this case m is 0.

In a preferred embodiment of the present invention n is 1 or dose to its lower limit of 1, such as 2 or 3. In particular n is 1 or 2, especially 1.

The radical of formula 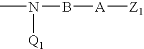
or the radical of formula 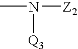
is attached directly to the carbon atom in the 2, 3 or 6-position, preferably in the 6-position, of the D-glucopyranosyl unit of the polysaccharide compound.

Advantageously the compounds of formulae (6), (9) and (10) are applied in excess to the compound of formula (5), such as a twofold, threefold, fourfold or fivefold molar excess.

Preferably the polysaccharide derivative of formula (1a) is prepared by reacting the compound of formula (5) with at least n molar equivalents of the compound of the formula (6) to yield the compound of formula (7), and allowing the compound of the formula (7) to react with at least n molar equivalents of the compound of the formula (8).

The compound of the formula (9) can be prepared by allowing approximately one molar equivalent of a compound of formula (6) to react with approximately one molar equivalent of a compound of formula (8) in a condensation reaction known per se.

Within the context of the present invention the leaving group precursor P* is understood to be a compound which is commonly used to chemically modify the hydroxyl group in order to increase its tendency to separate. Usually, this is carried out by introduction of e.g. an inorganic or organic acid radical P which is able to delocalize the binding electron pair. Examples for P are bromine, chlorine, p-toluene sulfonate (tosylate), p-bromobenzene sulfonate (brosylate), p-nitrobenzene sulfonate (nosylate), methane sulfonate (mesylate), trifluoromethane sulfonate (triflate), nonafluorobutane sulfonate (nonaflate) and 2,2,2-trifluoroethane sulfonate (tresylate). Such reactions are known in the field of organic chemistry and described in detail, for example, in March's Advanced Organic Chemistry, Reactions, Mechanisms, and Structure, M. B. Smith, J. March, John Wiley & Sons, 5^{th} Ed., 2001, 445.

There come into consideration as a leaving group precursor P*, for example, hydrogen bromide, thionyl chloride, p-toluene sulfonyl chloride, p-bromobenzene sulfonyl chloride (brosyl chloride), p-nitrobenzene sulfonyl chloride (nosyl chloride), methane sulfonyl chloride (mesyl chloride), trifluoromethane sulfonyl chloride, nonafluorobutane sulfonyl chloride and 2,2,2-trifluoroethane sulfonyl chloride (tresyl chloride), preferably toluene4-sulfonyl chloride.

X is for example, halogen, such as fluorine, chlorine or bromine, preferably chlorine,

Beside halogen, there also come into consideration for the leaving group X acyloxy radicals, such as acetyloxy or chloro acetyloxy.

Halogen is preferred for X.

Usually, the polysaccharide derivatives of formulae (1a) and (1b) and their precursor of formula (5) are mixtures of isomers, in particular, if n is larger than 1.

The compounds of the formulae (4), (6), (8) and (10) are known or can be obtained in a manner known per se. Compounds of formula (8) and (10) are described, for example, in the prior art pertaining to reactive dyestuffs.

PS corresponds to the backbone or skeleton of the polysaccharide compound of formula (5) apart from the hydroxy groups.

There come into consideration as the polysaccharide compound of formula (4) dextrin, cyclodextrin, alginic acid, alginic acid esters, chitin, chitosan, pectin, dextran and biopolymers containing oligosaccharide moieties, such as glycopeptides, preferably dextrin and cydodextrin and especially cydodextrin. In the context of the present invention the polysaccharide compound of formula (4) preferably corresponds to an oligosaccharide, i.e. compounds of a moderate molecular weight, which are water soluble, having in average e.g. up to 20 recurring units in the molecule.

The polysaccharide compound of formula (4) in the meaning of cyclodextrin comprises cyclodextrin and cyclodextrin derivatives which have at least one free hydroxy group in the cyclodextrin molecule in at least one of the 2, 3 or 6-position of the D-glucopyranosyl ring. There comes into consideration as the compound of formula (4) α-, β-, γ- or δ-cyclodextrin or higher cyclodextrins or a mixture thereof as well as the corresponding derivatives.

Examples of cydodextrin derivatives suitable for the preparation process according to the present invention are cydodextrin ethers or mixed ethers, cyclodextrin esters or mixed esters or mixed cyclodextrin/ether/ester derivatives, in particular said derivatives of β-cyclodextrin.

Hydrophilic cyclodextrin derivatives having the following substituents are suitable: a C₁-C₄alkyl radical, such as methyl or ethyl, preferably methyl; a C₂-C₆hydroxyalkyl radical, such as 2-hydroxyethyl, 2- or 3-hydroxypropyl or hydroxybutyl, preferably 2- or 3-hydroxypropyl, a C₃-C₆oligohydroxyalkyl radical, preferably a C₃-C₄oligohydroxyalkyl radical, particularly preferably a dihydroxypropyl radical, such as 2,3-dihydroxypropyl, an acetyl radical, a propionyl radical, a butyryl radical, preferably an acetyl radical, or a propionyl radical, particularly preferably an acetyl radical. Hydrophilic cyclodextrin derivatives having an average degree of substitution per anhydroglucose (DS) of, for example, 0.3-2.0, preferably of 0.6-1.8, come into consideration.

Ionic cydodextrin derivatives having the following substituents are also suitable: a C₁-C₄carboxyalkyl radical, such as carboxymethyl or 2-carboxyethyl, in the form of the free acid or as an alkali metal salt, a C₁-C₄alkyl radical substituted by sulfo, such as 2-sulfoethyl, in the form of the free acid or as an alkali metal salt, a C₂-C₄carboxyhydroxyalkyl radical in the form of the free acid or as an alkali metal salt, a C₂-C₄hydroxyalkyl radical substituted by sulfo in the form of the free acid or as an alkali metal salt. The average degree of substitution per anhydroglucose (DS) in these cyclodextrin derivatives is, for example, 0.3-2.0, preferably 0.4-1.5, especially 0.4-0.6.

Ionic cyclodextrin derivatives having an oxalyl radical, malonyl radical, succinyl radical, glutaryl radical and/or adipyl radical as substituents are also suitable, having an average degree of substitution per anhydroglucose (DS) of, for example, 0.3 to 2.0, preferably 0.4-1.5, especially 0.4-0.8.

In a particular embodiment of the present invention the compound of formula (4) corresponds to α-, β- or γ-cyclodextrin or a mixture of α-, β- and γ-cyclodextrin, especially to β-cyclodextrin.

Preferred are polysaccharide derivatives of the formula (1a), wherein
A is —S— or 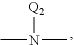
   preferably 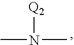
Q₁ is hydrogen, C₁-C₄alkyl, benzyl or the radical —B—A—Z₁, wherein A is as defined above and B and Z₁ are as defined hereafter,
Q₂ is hydrogen, C₁-C₄alkyl or benzyl, preferably hydrogen,
B is a C₂-C₆alkylene radical,
Z₁ is a radical of formula (2a), (2b), (2c) or (2d) as defined above,
   in which Y is vinyl, β-chloroethyl or β-sulfatoethyl, Hal is bromine, I is a number 2 or 3, X₁ is halogen, T₁ is C₁-C₄alkoxy, C₁-C₄alkylthio, hydroxy, amino, N-mono- or N,N-di-C₁-C₄alkylamino unsubstituted or substituted in the alkyl moiety by hydroxy, sulfato or by sulfo, morpholino, or phenylamino or N—C₁-C₄alkyl-N-phenylamino each unsubstituted or substituted in the phenyl ring by sulfo, carboxy, acetylamino, chlorine, methyl or by methoxy and wherein the alkyl is unsubstituted or substituted by hydroxy, sulfo or by sulfato, or naphthylamino unsubstituted or substituted by from 1 to 3 sulfo groups, or is a fibre-reactive radical of formula (3a′), (3b′), (3c′), (3d′) or (3f′) as defined above, especially (3c′) or (3d′) and in particular (3c′), in which
(R₄)₀₋₂ is 0 to 2 identical or different substituents from the group of methyl, methoxy and sulfo, especially methyl and methoxy,
Y is as defined above, and
Y₁ is a group —CH(Br)—CH₂—Br or —C(Br)═CH₂,
PS corresponds to the radical of a cyclodextrin or a cyclodextrin derivative,
m is 0, 1 or an integer greater than 1,
n is 1 or 2, preferably 1, and
   the sum of n+m corresponds to the original number of hydroxy groups in the reactive cydodextrin or cyclodextrin derivative of formula (1a), i.e. the total number of hydroxy groups in the cydodextrin or cyclodextrin derivative of formula (4).

Some of the compounds of formula (7) are new. Accordingly, the present invention relates also to the compound of formula (7), wherein PS, Q₁, A, B, m and n are as defined and preferred above, with the exception of β-cyclodextrin which is substituted in the 6-position of one of the D-glucopyranosyl units by 2-aminoethylenamino or 2-hydroxyethylenamino and γ-cyclodextrin which is substituted in the 6-position of one of the D-glucopyranosyl units by 2-aminoethylenamino.

The reactive polysaccharide derivatives according to the present invention are able to react with various compounds or substrates which contain nucleophilic groups capable to form a covalent bond upon reaction with the reactive radical Z, such as OH, NH or SH groups. Compounds which come into consideration are low molecular weight compounds, for example, alcohols, thiols or amines, or high molecular weight compounds, such as natural or synthetic polymers or a mixture of various polymer types, for example, starches, celluloses, glycogens, mannans, pectins, chitins, chitosans, alginic acid, albumins, collagen, elastin, globulins, fibrinogens, keratins, lignins, polyesters, polyamides, polyamines, phenolics, aminoplastics, polyurethanes, polyacrylic acids, polyacrylamides, polyallyl alcohols, polyallylamines, polyvinyl acetate polymers, polyvinyl alcohols, polyepoxides, cellulose-acrylates, starch-acrylates, biopolymers containing polysaccharide moieties, such as glycopeptides or starch protein and the like. Substrates which come into consideration comprise, for example, the polymers mentioned above which are substantially insoluble in water. They are, for example, in the form of pellets, beads, sheets or fibers. Examples are polymer beads, paper, textile fiber materials, keratinic fibers, such as human hair or leather. As possible substrates there come into consideration also self-assembled monolayers (SAMs) on silver or gold substrates bearing e.g. terminal hydroxyl, thiol or amino groups. SAMs are described, for example, in Science 1991, 254 (5036), 1312-1319; Journal of Physical Chemistry B, 1998, 102(2), 426-436; or WO-A-98/58 967. Modification of said substrates with the reactive polysaccharide derivatives of formula (1a) or (1b) affects, in particular, the surface or surface near regions.

Accordingly, the present invention relates also to a process for the preparation of compounds or substrates modified with polysaccharide, comprising reacting the said compounds or substrates with a polysaccharide derivative of formula (1a) or (1b), wherein PS, Q₁, Q₂, Q₃, B, Z₁, Z₂, m and n are as defined and preferred above.

The modification of high molecular weight compounds and the surface modification of substrates may be carried out, for example, in accordance with the methods described in U.S. Pat. No. 5,728,823.

The modified compounds or substrates thus obtained can be employed in numerous applications, such as given in U.S. Pat. No. 5,728,823.

Modified polymers or substrates may be used, e.g.:
to improve adhesion to surfaces,
to solubilize the polymer/oligomer in the corresponding matrix,
to render the polymer hydrophilic or hydrophobic,
to improve wettability and compatibility with the surrounding medium,
to increase stability to coagulation,
to modify rheology,
to improve film formation,
to complex active compounds, such as biocides, insecticides, acaricides, fungicides, herbicides, pheromones, fragrances, flavorings, pharmaceutical active compounds, active compounds for antistatic finishing or flame retardant finishing, UV-stabilizers, dyestuffs or a mixture thereof in the cydodextrin cavity and release the active compounds in a controlled manner,
to increase the bioavailability of active compounds,
to stabilize active compounds, e.g. to light, temperature, oxidation, hydrolysis, evaporation by complex formation,
to solubilize active compounds,
to extract active compounds from gaseous or liquid media,
as a filter system, i.e. to absorb undesired substances from gaseous or liquid media,
as a collector system, i.e. to absorb desired substances from gaseous or liquid media,
as selective separating media in chromatography,
decrease toxicity or irritation of active compounds by complex formation,
to absorb, complex or encapsulate substances causing unpleasant or toxic odors, e.g. degradation products, by-products of polymers etc., such as perspiration products, acetic acid, butyric acid, amines, sulfur compounds or residual monomers of toxic substances,
as formulation aid for active ingredients, e.g. as a powdered formulation.

Preferred as the substrates are textile fiber materials containing hydroxyl groups or containing nitrogen or paper, in particular textile fiber materials. Textile fiber materials can be in the form of fiber, yam or piece goods, such as non-wovens, knitted and woven goods, pile fabrics or terry goods. Examples are silk, wool, polyamide fibers and polyurethanes, and in particular all types of cellulosic fiber materials. Such cellulosic fiber materials are, for example, the natural cellulosic fibers, such as cotton, linen and hemp, as well as cellulose and regenerated cellulose. The reactive polysaccharide derivatives according to the invention are also suitable for finishing fibers containing hydroxy groups which are contained in blend fabrics, for example mixtures of cotton with polyester fibers or polyamide fibers. The reactive polysaccharide derivatives according to the invention are particularly suitable for finishing cellulosic materials. They can furthermore be used for finishing natural or synthetic polyamide fiber materials.

The reactive polysaccharide derivatives of formulae (1a) and (1b) are applied to the textile goods in aqueous solution, in analogy to the dyeing processes known for reactive dyes or finishing processes in textile industry. They are suitable both for the exhaust- and for the pad-method, in which the goods are impregnated with aqueous solutions, which may contain salts. Dyeing machines customary in dyeing with reactive dyes are preferably utilized for this. The reactive polysaccharides are fixed, if appropriate after an alkali treatment, or preferably in the presence of alkali, under the action of heat, steam or by storage at room temperature for several hours, thereby forming a chemical bond with the substrate. The reactive polysaccharide derivatives according to the invention can also be applied in the presence of crosslinking agents or resin finish, for example, dimethylol-urea, dimethoxy-methyl-urea, trimethoxy-methyl-melamin, tetramethoxy-methyl-melamine, hexamethoxy-methyl-melamine, dimethylol-dihydroxy-ethylene-urea, dimethylol-propylene-urea, dimethylol-4-methoxy-5,5′-dimethyl-propylene-urea, dimethylol-5-hydroxypropylene-urea, butane-tetra-carboxylic-acid, citric acid, maleic acid, bonding agents, for example, acrylates, silicones, urethanes, butadienes, in a textile finishing process which may result in superior effect durability. Such textile finishing processes are described, for example, in DE-A-40 35 378. After the fixing, the finished substrates are rinsed thoroughly with cold and hot water, if appropriate with the addition of an agent which has a dispersing action and promotes diffusion of the non-fixed portions.

The finished substrates contain, for example, 0.1 to 25% by weight, preferably 1 to 10% by weight, of the reactive polysaccharide derivative according to the present invention, based on the total weight of the substrate.

The finished substrates can be used to complex or encapsulate, for example, UV-stabilizers, antimicrobials, biocides, bactericides, acaricides, insecticides, fungicides, pharmaceutical active compounds, fragrances, perfumes, pheromones, vitamines or skin-, hair and textile benefit agents, e.g. UV-absorber, fatty acids, anti-irritants or inflammatory agents, to e.g. solubilize water-insoluble or poorly water-soluble substances, to increase the bioavailability of active compounds; to stabilize substances against light, temperature, oxidation, hydrolysis or from volatility, to mask bad taste or unpleasant odor, to slowly release active compounds in a controlled manner over a prolonged period of time (delivery systems). On the other side, the finished substrates are useful to assimilate chemical substances, e.g. from a gaseous or liquid environment, which are captured, e.g. in the cydodextrin cavity, thereby serving as a collector system. Such collector systems may find application in the field of medical diagnostics, help to determine pollutants from the environment or depollute or decontaminate gaseous of liquid media. Decomposition products of sweat are trapped in the cyclodextrin cavity, thus diminishing or preventing malodor. Textile materials, such as clothings finished with the inventive composition stay fresh with a pleasant smell. Laundering ensures removal of the decomposition products of sweat from the cydodextrin cavity and regeneration of the system.

The Examples given hereinbelow are intended to illustrate the invention without limiting it to the Examples specifically mentioned.

### EXAMPLE 1

Monα-(6-O-p-toluenesulfonyl)-β-cyclodexbrin is prepared according to procedures known in the art such as described in Synthetic Communications, 25 (5), 703-710 (1995) and Inorganic Chimica Acta 272 (1998), 162-167.

### EXAMPLE 2

A mixture of 5 gram of mono-(6-O-β-toluenesulfonyl)-β-cyclodextrin prepared according to Example 1 in 180 milliliter of N-ethylethylendiamin (1-ethylamino-2-aminoethan) is stirred at 100° C. for 3 hours. After cooling and slow addition of 200 milliliter of water, the resulting solution is poured into 3000 milliliter of aceton. The suspension thus obtained is filtered, rinsed with aceton and dried at 50° C. in a vacuum oven to yield 3.5 gram of a cyclodextrin derivative as a white powder (mass spectroscopy m/e=1206), which corresponds to the formula 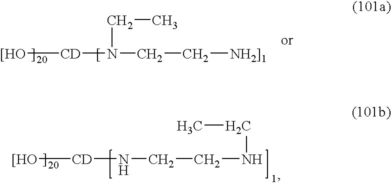
wherein CD is the carbon backbone of β-cyclodextrin and the diamino radical is bonded to the 6-position of the D-glucopyranosyl ring.

Proton NMR DMSO-d₆: δ (ppm) 5.60 (broad s), 4.79 (sharp d, J=5.9 Hz), 3.40-3.80 (m), 3.15-3.45 (m), 2.76-2.85 (—CH₂—, m), 2.40-2.75 (—CH₂—, m), 0.94 (—CH₃, t).

### EXAMPLE 3

A mixture of 0.3 gram of mono-(6-O-p-toluenesulfonyl)-β-cyclodextrin prepared according to Example 1 in 10 milliliter of N-phenylethylendiamin (1-phenylamino-2-aminoethan) is stirred at 115° C. for 3 hours. The resulting mixture is allowed to cool and poured into 300 milliliter of aceton. The suspension thus obtained is filtered, rinsed with aceton and dried at 50° C. in a vacuum oven to yield 0.1 gram of a cyclodextrin derivative as a beige powder, which corresponds to the formula 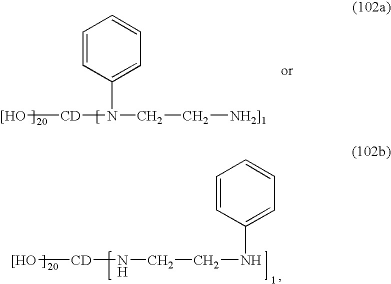
wherein CD is the carbon backbone of β-cyclodextrin and the diamino radical is bonded to the 6position of the D-glucopyranosyl ring.

Methylen signals are detected in the proton NMR spectrum measured in DMSO-d₆ at chemical shifts 2.75-2.80 and 2.92-2.98 ppm.

### EXAMPLE 4

A mixture of 0.3 gram of mono-(6-O-p-toluenesulfonyl)-β-cyclodextrin prepared according to Example 1 in 10 milliliter of N-isopropylethylendiamin (1-isopropylamino-2-aminoethan) is stirred at 115° C. for 3 hours. The resulting mixture is allowed to cool and poured into 400 milliliter of aceton. The suspension thus obtained is filtered, rinsed with aceton and dried at 50° C. in a vacuum oven to yield 0.1 gram of a cyclodextrin derivative as a beige powder, which corresponds to the formula 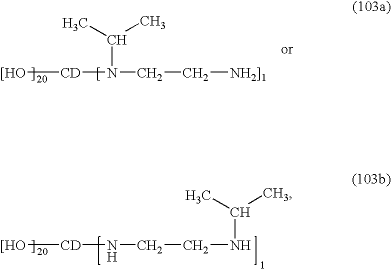
wherein CD is the carbon backbone of β-cyclodextrin and the diamino radical is bonded to the 6-position of the D-glucopyranosyl ring.

Proton NMR DMSO-d₆: δ (ppm) 4.80 (sharp d), 3.45-3.75 (m), 3.20-3.43 (m), 2.40-2.70 (—CH₂— and —CH—, m), 0.91-0.99 (—CH₃).

### EXAMPLE 5

A mixture of 0.3 gram of mono-(6-O-p-toluenesulfonyl)-β-cyclodextrin prepared according to Example 1 in 90 milliliter of diethylentriamin is stirred at 115° C. for 3 hours. The resulting mixture is allowed to cool and poured into 1000 milliliter of aceton. The suspension thus obtained is filtered, rinsed with aceton and dried at 50° C. in a vacuum oven to yield 0.18 gram of a cyclodextrin derivative as a white powder, which corresponds to the formula 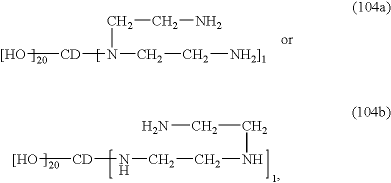
wherein CD is the carbon backbone of β-cyclodextrin and the trisamino radical is bonded to the 6-position of the D-glucopyranosyl ring.

Proton NMR DMSO-d₆: δ (ppm) 4.80 (sharp d), 3.70-3.95 (m), 3.40-3.70 (m), 3.20-3.40 (m), 2.70-2.95 (—CH₂—, m), 2.30-2.70 (—CH₂—, m).

### EXAMPLE 6

A mixture of 0.3 gram of mono-(6-O-p-toluenesulfonyl)-β-cyclodextrin prepared according to Example 1 in 10 milliliter of N-propylethylendiamin (1-propylamino-2-aminoethan) is stirred at 115° C. for 3 hours. The resulting mixture is allowed to cool and poured into 400 milliliter of aceton. The suspension thus obtained is filtered, rinsed with aceton and dried at 50° C. in a vacuum oven to yield 0.27 gram of a cyclodextrin derivative as a white powder, which corresponds to the formula 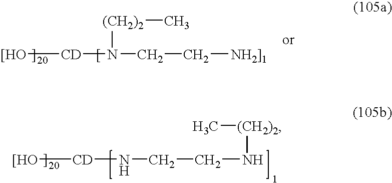
wherein CD is the carbon backbone of β-cyclodextrin and the diamino radical is bonded to the 6position of the D-glucopyranosyl ring.

Methylen signals are detected in the proton NMR spectrum measured in DMSO-d₆ at chemical shifts 2.60-2.90 ppm.

### EXAMPLE 7

A mixture of 1.0 gram of mono-(6-O-p-toluenesulfonyl)-β-cyclodextrin prepared according to Example 1 in 10 milliliter of N-benzylethylendiamin (1-benzylamino-2-aminoethan) is stirred at 115° C. for 3 hours. The resulting mixture is allowed to cool and poured into 400 milliliter of aceton. The suspension thus obtained is filtered, rinsed with aceton and dried at 50° C. in a vacuum oven to yield 0.94 gram of a cyclodextrin derivative as an off-white powder, which corresponds to the formula 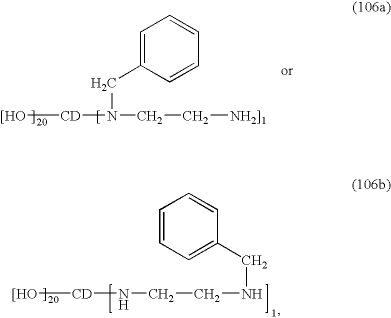
wherein CD is the carbon backbone of β-cyclodextrin and the diamino radical is bonded to the 6-position of the D-glucopyranosyl ring.

Methylen signals are detected in the proton NMR spectrum measured in DMSO-d₆ at chemical shifts 2.62-2.90 ppm.

### EXAMPLE 8

A mixture of 0.3 gram of mono-(6-O-p-toluenesulfonyl)-β-cyclodextrin prepared according to Example 1 in 30 milliliter of N-hydroxyethylethylendiamin (1-[2-hydroxyethyl]amino-2-aminoethan) is stirred at 115° C. for 3 hours. The resulting mixture is allowed to cool and poured into 500 milliliter of aceton. The suspension thus obtained is filtered, rinsed with aceton and dried at 50° C. in a vacuum oven to yield 0.16 gram of a cyclodextrin derivative as a white powder, which corresponds to the formula 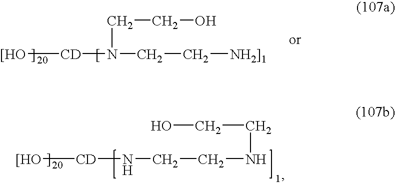
wherein CD is the carbon backbone of β-cyclodextrin and the diamino radical is bonded to the 6-position of the D-glucopyranosyl ring.

Methylen signals are detected in the proton NMR spectrum measured in DMSO-d₆ at chemical shifts 2.60-2.92 ppm.

### EXAMPLE 9

A mixture of 0.3 gram of mono-(6-O-p-toluenesulfonyl)-β-cyclodextrin prepared according to Example 1 in 30 milliliter of 2-ethylaminoethanol is stirred at 115° C. for 3 hours. The resulting mixture is allowed to cool and poured into 500 milliliter of aceton. The suspension thus obtained is filtered, rinsed with aceton and dried at 50° C. in a vacuum oven to yield 0.13 gram of a cyclodextrin derivative as a white powder, which corresponds to the formula 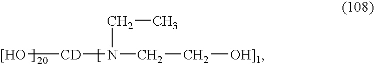
wherein CD is the carbon backbone of β-cyclodextrin and the amino radical is bonded to the 6-position of the D-glucopyranosyl ring.

### EXAMPLE 10

A mixture of 0.3 gram of mono-(6-O-p-toluenesulfonyl)-β-cyclodextrin prepared according to Example 1 in 30 milliliter of N-methylethylendiamin (1-methylamino-2-aminoethan) is stirred at 115° C. for 3 hours. After cooling, the resulting solution is poured into 300 milliliter of aceton. The suspension thus obtained is filtered, rinsed with aceton and dried at 50° C. in a vacuum oven to yield 0.13 gram of a cyclodextrin derivative as a white powder which corresponds to the formula 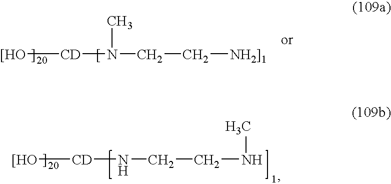
wherein CD is the carbon backbone of β-cyclodextrin and the diamino radical is bonded to the 6-position of the D-glucopyranosyl ring.

Methylen signals are detected in the proton NMR spectrum measured in DMSO-₆ at chemical shifts 2.20-2.30, 2.45-2.65 and 2.80-2.90 ppm.

### EXAMPLE 11

A mixture of 0.3 gram of mono-(6-O-p-toluenesulfonyl)-β-cyclodextrin prepared according to Example 1 in 10 milliliter of N-butylethylendiamin (1-butylamino-2-aminoethan) is stirred at 115° C. for 3 hours. After cooling, the resulting solution is poured into 200 milliliter of aceton. The suspension thus obtained is filtered, rinsed with aceton and dried at 50° C. in a vacuum oven to yield 0.03 gram of a cyclodextrin derivative as a white powder which corresponds to the formula 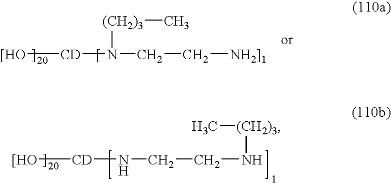
wherein CD is the carbon backbone of β-cyclodextrin and the diamino radical is bonded to the 6-position of the D-glucopyranosyl ring.

Methylen signals are detected in the proton NMR spectrum measured in DMSO-d₆ at chemical shifts 2.25-2.65 and 2.80-2.90 ppm.

### EXAMPLE 12

A mixture of 0.3 gram of mono-(6-O-p-toluenesulfonyl)-β-cyclodextrin prepared according to Example 1 in 15 milliliter of N-methylpropylendiamin (1-methylamino-3-aminopropan) is stirred at 115° C. for 3 hours. After cooling, the resulting solution is poured into 400 milliliter of aceton. The suspension thus obtained is filtered, rinsed with aceton and dried at 50° C. in a vacuum oven to yield 0.27 gram of a cydodextrin derivative as a white powder which corresponds to the formula 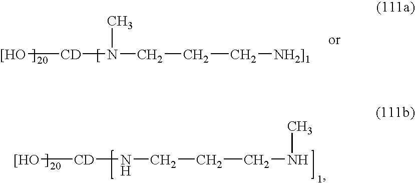
wherein CD is the carbon backbone of β-cyclodextrin and the diamino radical is bonded to the 6position of the D-glucopyranosyl ring.

Methylen signals are detected in the proton NMR spectrum measured in DMSO-d₆ at chemical shifts 2.50-2.90 ppm.

### EXAMPLE 13

A mixture of 10 gram of mono-(6-O-p-toluenesulfonyl)-β-cyclodextrin prepared according to Example 1 in 100 gram of diethanolamin is stirred at 115° C. for 3 hours. The resulting mixture is allowed to cool and poured into 1000 milliliter of an aceton/water mixture (5/1). The suspension thus obtained is filtered, rinsed with aceton and dried at 50° C. in a vacuum oven to yield 7.54 gram of a cyclodextrin derivative as a white powder, which corresponds to the formula 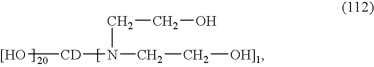
wherein CD is the carbon backbone of β-cyclodextrin and the amino radical is bonded to the 6-position of the D-glucopyranosyl ring.

Proton NMR DMSO-d₆: δ (ppm) 4.70-4.82 (sharp m), 3.70-3.80 (sharp m), 3.45-3.65 (m), 3.23-3.42 (m), 3.12 (—CH₂—, t), 2.80-2.90 (—CH₂—, m), 2.40-2.62 (—CH₂—, m). Thermogravimetric analysis: mid-point temperature is 305.7° C.

### EXAMPLE 14

In a manner analogous to that described in Example 2 it is possible to obtain a cyclodextrin derivative which corresponds to the formula 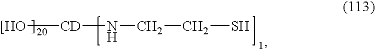
wherein CD is the carbon backbone of β-cyclodextrin and the amino radical is bonded to the 6-position of the D-glucopyranosyl ring, if 2-aminoethylmercaptan is used in place of N-ethylethylendiamin.

### EXAMPLE 15

In a manner analogous to that described in Example 2 it is possible to obtain a cyclodextrin derivative which corresponds to the formula 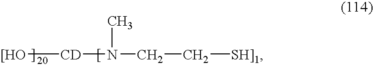
wherein CD is the carbon backbone of β-cyclodextrin and the amino radical is bonded to the 6-position of the D-glucopyranosyl ring, if 2-(N-methylamino)ethylmercaptan is used in place of N-ethylethylendiamin.

### EXAMPLE 16a

0.4 gram of the compound obtained according to Example 2 are dissolved in 60 milliliter of water and the pH of the solution is adjusted to 6 using 1 N hydrochloric acid. 0.07 gram of the compound of formula 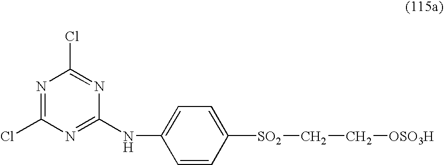
are then added dropwise at room temperature simultaneously with aqueous sodium carbonate (15%) which is automically added via a dosimat apparatus to maintain a pH around 6.5. After 2.5 hours the reaction mixture is filtered and the filtrate is poured into 1000 milliliter of aceton. The suspension thus obtained is filtered and dried at 50° C. in a vacuum oven to yield 0.37 gram of a cyclodextrin derivative as a white powder (mass spectroscopy m/z=1596), which corresponds to the formula 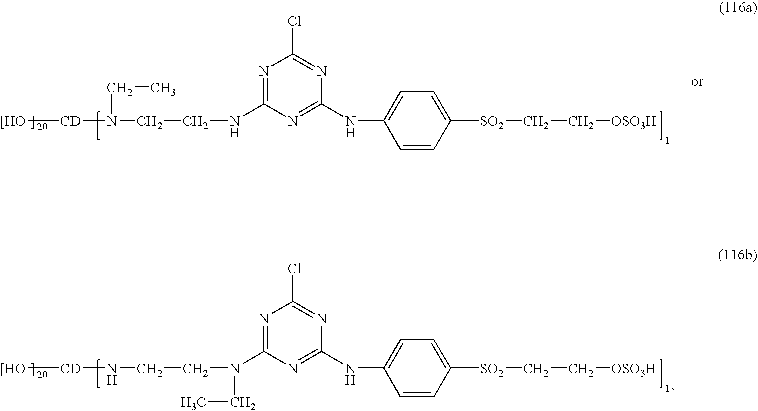
wherein CD is the carbon backbone of β-cyclodextrin and the reactive radical is bonded to the 6-position of the D-glucopyranosyl ring.

Proton NMR DMSO-d₆: δ (ppm) 7.60-7.90 (phenyl, m), 5.60 (broad s), 4.20-4.60 (broad m), 3.70-3.80 (sharp m), 3.80-4.00 (—CH₂—, m), 3.00-3.80 (m), 2.70-2.95 (—CH₂—, m).

The compound of formula (115a) is prepared according to procedures known in the art of reactive dyestuff preparation by condensation of cyanurchloride with sulfuric acid mono-[2-(4-amino-benzenesulfonyl)-ethyl] ester.

Valuable reactive cyclodextrin derivatives can likewise be prepared in a manner analogous to that described in Example 16a, if the compound of formula (101a) or (101b) obtained according to Example 2 is replaced by an equimolar amount of one of the compounds of the formulae (102a) or (102b), (103a) or (103b), (104a) or (104b), (105a) or (105b), (106a) or (106b), (107a) or (107b), (108), (109a) or (109b), (110a) or (111b), (111a) or (111b), (112), (113) and (114) obtained according to Examples 3 to 15.

### EXAMPLE 16b

6 gram of the compound obtained according to Example 2 are dissolved in 200 milliliter of water and the pH of the solution is adjusted to 6.5 using 1N hydrochloric acid. 2.14 gram of the compound of formula (115a) are then added dropwise as an aqueous suspension (45 gram total) at room temperature simultaneously with aqueous sodium carbonate as is described above in Example 16a. After 3 hours the reaction mixture is filtered (porosity 4) and the filtrate is poured into 1400 milliliter of aceton. The white suspension is in turn filtered and dried at 50° C. in a vacuum oven to yield 7.3 gram of product corresponding to formula (116a) or (116b).

The proton NMR spectrum of the above obtained product measured in DMSO-d₆ is identical to that obtained in Example 16a.

Elemental analysis: calculated C, 39.49; H, 5.95; N, 4.85; Cl, 2.04; O, 43.92; S, 3.70; experimental C, 40.76; H, 5.90; N, 4.54; Cl, 2.82; O, 40.81; S, 3.49.

### EXAMPLES 17 TO 46

Valuable Reactive Cyclodextrin Derivatives which Essentially Comprise a Compound of the Following General Formula

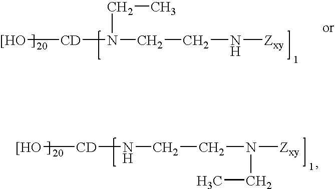

in which Z_{xy} in each case are the radicals listed in the 3^{rd} column of Table 1 can likewise be prepared in a manner analogous to that described in Example 16, if the compound of formula (115) is replaced by an equimolar amount of one of the compounds of the formulae Z_{xy}—Cl given in the 2^{nd} column of table 1.

TABLE 1

| Exp. | Z_{xy}—Cl | Z_{xy} |
|---|---|---|
| 17 | Z₁₁—Cl | 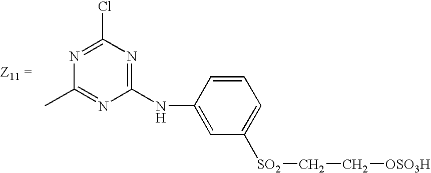 |
| 18 | Z₁₂—Cl | 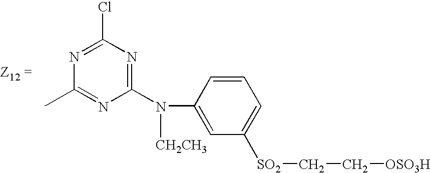 |
| 19 | Z₁₃—Cl | 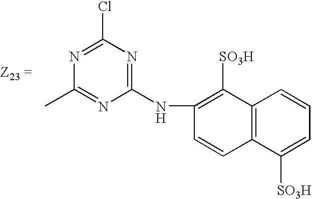 |
| 20 | Z₁₄—Cl | 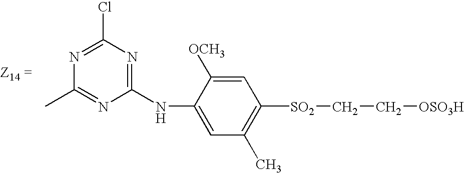 |
| 21 | Z₁₅—Cl | 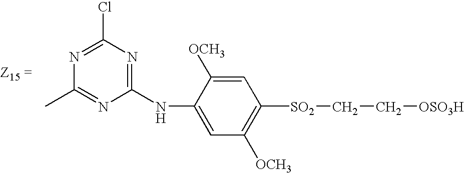 |
| 22 | Z₁₆—Cl | 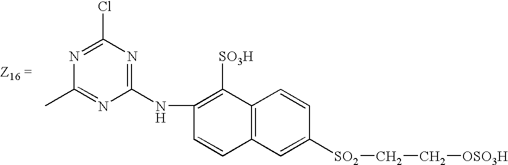 |
| 23 | Z₁₇—Cl | 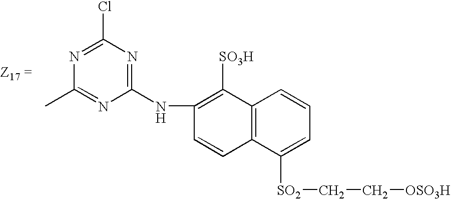 |
| 24 | Z₁₈—Cl | 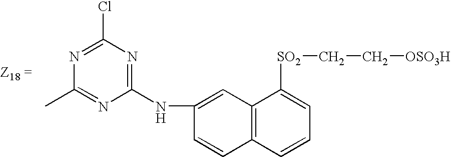 |
| 25 | Z₁₉—Cl | 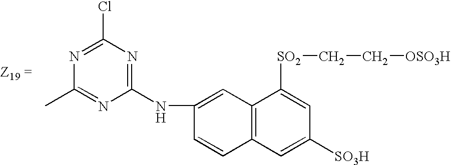 |
| 26 | Z₂₀—Cl | 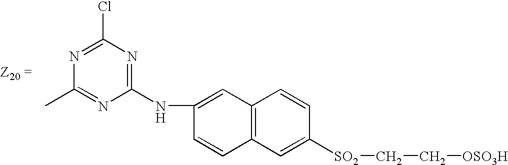 |
| 27 | Z₂₁—Cl | 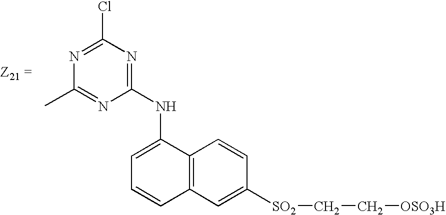 |
| 28 | Z₂₂—Cl | 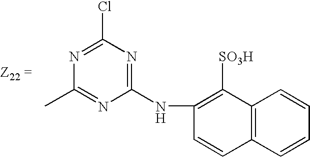 |
| 29 | Z₂₃—Cl | 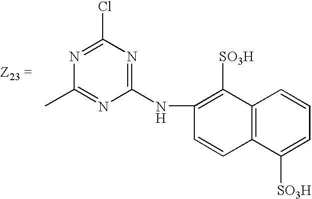 |
| 30 | Z₂₄—Cl | 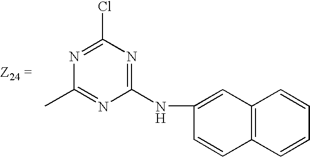 |
| 31 | Z₂₅—Cl | 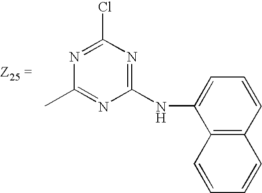 |
| 32 | Z₂₆—Cl | 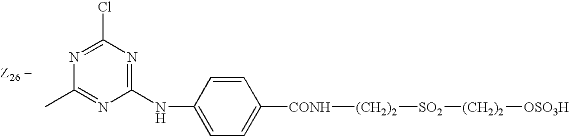 |
| 33 | Z₂₇—Cl | 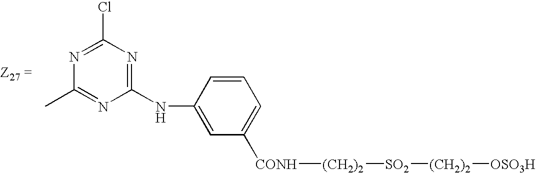 |
| 34 | Z₂₈—Cl | Z₂₈ = —CO—(CH₂)₃—SO₂—(CH₂)₂—Cl |
| 35 | Z₂₉—Cl | 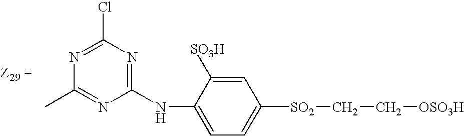 |
| 36 | Z₃₀—Cl | 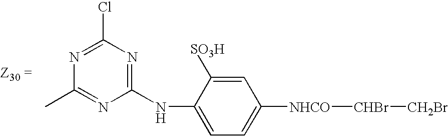 |
| 37 | Z₃₁—Cl | 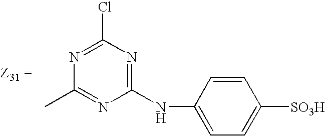 |
| 38 | Z₃₂—Cl | 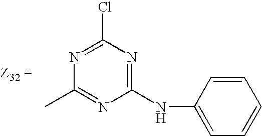 |
| 39 | Z₃₃—Cl | 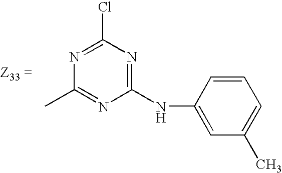 |
| 40 | Z₃₄—Cl | 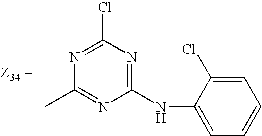 |
| 41 | Z₃₅—Cl | 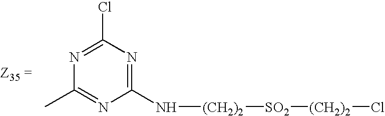 |
| 42 | Z₃₆—Cl | 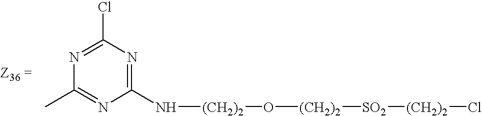 |
| 43 | Z₃₇—Cl | 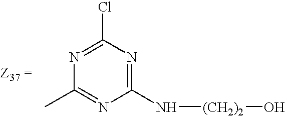 |
| 44 | Z₃₈—Cl | 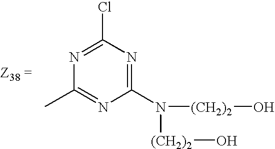 |
| 45 | Z₃₉—Cl | 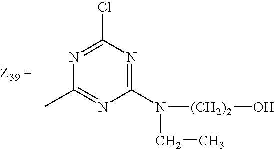 |
| 46 | Z₄₀—Cl | 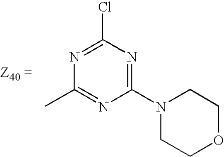 |

### EXAMPLE 47

0.3 gram of sulfuric acid mono-[2-(3-amino-benzenesulfonyl)-ethyl] ester and 1.5 gram of mono-(6-O-p-toluenesulfonyl)-β-cyclodextrin obtained according to example 1 are dissolved in 20 milliliter of pyridin. The mixture is heated and stirred at 55° C. for 1.5 hour and then at 100° C. for 2.5 hours. After cooling to 30° C., the reaction mixture is filtered and dried in a vacuum oven at 50° C. to yield 0.34 gram of a white powder. The filtrate is then poured into aceton. The precipitate is filtered and dried in a vacuum oven at 50° C. to yield 0.61 gram of a beige powder. The product corresponds to the formula 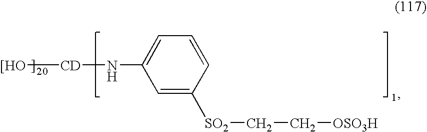
wherein CD is the carbon backbone of β-cyclodextrin and the reactive radical is bonded to the 6-position of the D-glucopyranosyl ring.

### EXAMPLE 48

0.3 gram of sulfuric acid mono-[2-(4-amino-benzenesulfonyl)ethyl] ester and 1.5 gram of mono-6-O-p-toluenesulfonyl)-β-cyclodextrin obtained according to example 1 are dissolved in 20 milliliter of pyridin. The mixture is heated and stirred at 55° C. for 1.5 hour and then at 100° C. for 2.5 hours. After cooling to 30° C., the reaction mixture is filtered and dried in a vacuum oven at 50° C. to yield 0.35 gram of a white powder. The filtrate is then poured into aceton. The precipitate is filtered and dried in a vacuum oven at 50° C. to yield 0.71 gram of as a beige powder. The product corresponds to the formula 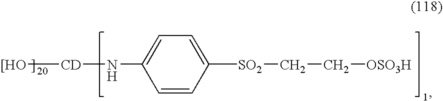
wherein CD is the carbon backbone of β-cyclodextrin and the reactive radical is bonded to the 6-position of the D-glucopyranosyl ring.

### EXAMPLE 49

0.37 gram of sulfuric acid mono-[2-(3-ethylamino-benzenesulfonyl)-ethyl] ester and 1.5 gram of mono-(6-O-p-toluenesulfonyl)-β-cyclodextrin obtained according to example 1 are dissolved in 20 milliliter of pyridin. The mixture is heated and stirred at 55° C. for 1.5 hour and then at 100° C. for 2.5 hours. After cooling to 30° C., the reaction mixture is filtered and dried in a vacuum oven at 50° C. to yield 0.19 gram of a white powder. The filtrate is then poured into aceton. The precipitate is filtered and dried in a vacuum oven at 50° C. to yield 1.15 gram of a beige powder. The product corresponds to the formula 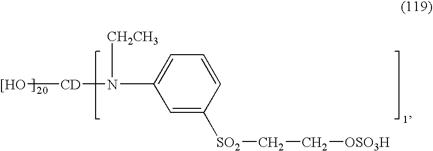
wherein CD is the carbon backbone of β-cyclodextrin and the reactive radical is bonded to the 6position of the D-glucopyranosyl ring.

### EXAMPLE 50

1.7 gram of the compound obtained according to Example 2 are dissolved in 50 milliliter of water and the pH of the solution is adjusted to 6.8 using 4N hydrochloric acid. 1.90 gram of the compound of formula 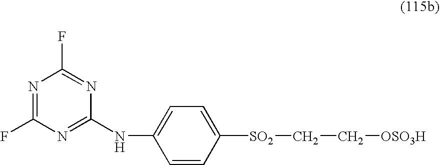
are then added dropwise as an aqueous suspension (32.6 gram total) at room temperature simultaneously with aqueous sodium carbonate as is described above in Example 16a. The reaction mixture is kept stirring at pH around 6 for 14 hours. The turbid mixture is then filtered (porosity 4) and the filtrate is poured into 500 milliliter of ethanol. The white suspension is in turn filtered and dried at 60° C. in a vacuum oven to yield 1.14 gram of product corresponding to formula 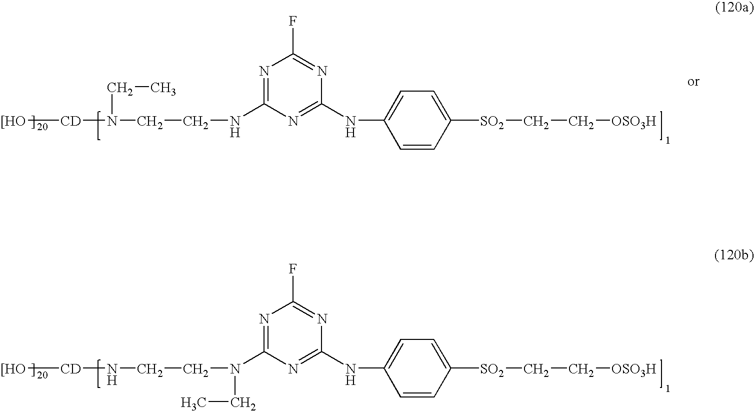
wherein CD is the carbon backbone of β-cyclodextrin and the reactive radical is bonded to the 6-position of the D-glucopyranosyl ring.

Proton NMR DMSO-d₆: δ (ppm) 7.40-7.96 (phenyl, m), 5.20-5.95 (m), 4.60-5.93 (broad m), 4.02-4.57 (m), 3.70-3.95 (s), 3.40-3.72 (m), 2.80-3.37 (m), 0.90 (broad m)

The compound of formula (115b) is prepared according to procedures known in the art of reactive dyestuff preparation by condensation of trifluorotriazine with sulfuric acid mono-[2-(4-amino-benzenesulfonyl)ethyl] ester.

### EXAMPLE 51

10 gram of the compound obtained according to Example 5 are dissolved in 100 milliliter of water and the pH of the solution is adjusted to 7 using 4N hydrochloric acid. 6.88 gram of the compound of formula (115a) are then added dropwise as an aqueous suspension (75 gram total) at room temperature simultaneously with aqueous sodium carbonate as is described above in Example 16a. After 2.5 hours the reaction mixture is filtered (porosity 4) and the filtrate is poured into 2000 milliliter of aceton. The off-white suspension is in turn filtered and dried at 60° C. in a vacuum oven to yield 14.2 gram of product corresponding to formula 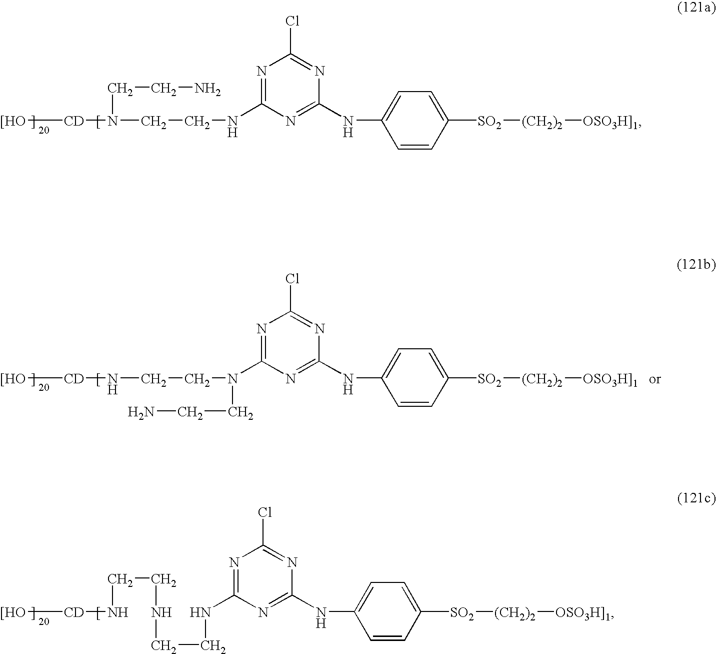
wherein CD is the carbon backbone of β-cyclodextrin and the reactive radical is bonded to the 6-position of the D-glucopyranosyl ring.

Mass spectroscopy: m/z [M(121)-2H+3Na]=2072.4, m/z [M(121)-H+2Na]=1656.4.

Proton NMR DMSO-d₆: δ (ppm) 7.40-7.90 (phenyl, m), 5.40-5.80 (broad s), 4.60-4.90 (s), 4.20-4.55 (broad s), 3.80-4.05 (—CH₂—, m), 3.00-3.80 (m), 2.60-3.10 (—CH₂—, m).

### EXAMPLES 52 TO 81

Valuable Reactive Cyclodextrin Derivatives which Essentially Comprise a Compound of the Following General Formula 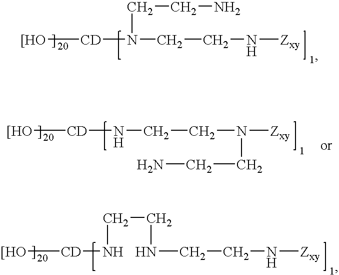
in which Z_{xy} in each case are the radicals listed in the 3^{rd} column of Table 1 given above can likewise be prepared in a manner analogous to that described in Example 51, if the compound of formula (115a) is replaced by an equimolar amount of one of the compounds of the formulae Z_{xy}—Cl given in the ₂^{nd} column of table 1 given above. Compounds of the formulae indicated above may be obtained, wherein two or three reactive radicals Z_{xy} are attached to the vacant binding sites of the nitrogen atoms in the same molecule.

### EXAMPLE 82

5 gram of the compound obtained according to Example 8 are dissolved in 200 milliliter of water and the pH of the solution is adjusted to 6.5 using hydrochloric acid. 1.80 gram of the compound of formula (115a) are then added dropwise as an aqueous suspension (27 gram total) at room temperature simultaneously with aqueous sodium carbonate as is described above in Example 16a. After 2 hours the reaction mixture is filtered (porosity 4) and the filtrate is poured into 1500 milliliter of aceton. The thus obtained liquor is allowed to decant overnight and filtered (porosity 4). The presscake is dried at 50° C. in a vacuum oven to yield 2.15 gram of a beige powder corresponding to formula 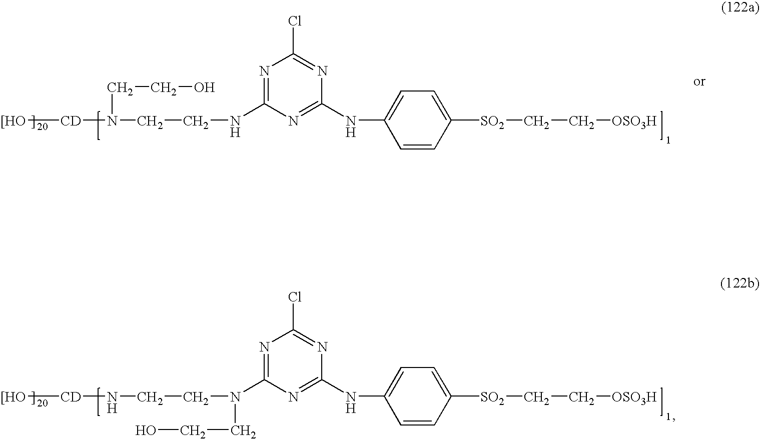
wherein CD is the carbon backbone of β-cyclodextrin and the reactive radical is bonded to the 6-position of the D-glucopyranosyl ring.

Proton NMR DMSO-d₆: δ (ppm) 7.83-8.02 (phenyl, m), 7.50-7.82 (phenyl, m), 5.65 (broad s), 4.80 (sharp s), 4.20-4.60 (broad s), 3.40-3.80 (m), 3.10-3.39 (m), 2.50-2.90 (m).

### APPLICATION EXAMPLES

### Example A1

Bath Recipe for Pad-Dry-Thermofix Application:

40.5 milliliter of a bath are prepared containing 2.22 gram of the compound of Example 16, 4.0 gram of urea, 0.41 gram of anhydrous sodium carbonate and 36.25 gram of water having a pH of 10.7.

### Example A2

Pad-Dry (120° C.) Thermofix (150° C.) on Bleached Cretonne:

5.83 gram of a cotton fabric (bleached cretone 135 g/m²) is padded at room temperature (22° C.) with the bath obtained according to Example A1. The pick up ratio is 71.0%. After drying for 60 sec at 120° C. in a drying tenter, the fabric is thermofixed in the same apparatus for 60 sec at 150° C. Unreacted material, urea and salts are removed by rinsing 5 times with water at 20° C. for 10 min until the pH is stable at around 8 (bath ratio 1/90). The fabric is then air dried. Accurate mass measurements before and after processing indicate an uptake of 2.9% of reacted cydodextrin based on the weight of the fabric.

Diagnostic test by discolouration of a basic purple phenolphthaleine solution reveals the presence of reacted cyclodextrin on cotton before and after machine wash at 50° C. with detergent ECE77.

### Example A3

Pad-Dry (120° C.) Thermofix (180° C.) on Bleached Cretonne:

5.87 gram of a cotton fabric (bleached cretonne; 135 g/m²) is padded at room temperature (22° C.) with the bath obtained according to Example A1. The pick up ratio is 70.4%. After drying for 60 sec at 120° C. in a drying tenter, the fabric is thermofixed in the same apparatus for 60 sec at 180° C. Unreacted material, urea and salts are removed by rinsing 5 times with water at 20° C. for 10 min until the pH is stable at around 8 (bath ratio 1/90). The fabric is then air dried. Accurate mass measurements before and after processing indicate an uptake of 3.7% of reacted cydodextrin based on the weight of the fabric.

Diagnostic test by discolouration of a basic purple phenolphthaleine solution reveals the presence of reacted cyclodextrin on cotton before and after machine wash at 50° C. with detergent ECE77.

### Example A4

Pad-Dry (120° C.) Thermofix (150° C.) on Bleached Mercerized Cretonne:

5.78 gram of a cotton fabric (bleached mercerized cretonne; 140 g/m²) is padded at room temperature (22° C.) with the bath obtained according to Example A1. The pick up ratio is 68.5%. After drying for 60 sec at 120° C. in a drying tenter, the fabric is thermofixed in the same apparatus for 60 sec at 150° C. Unreacted material, urea and salts are removed by rinsing 5 times with water at 20° C. for 10 min until the pH is stable at around 8 (bath ratio 1/90). The fabric is then air dried. Accurate mass measurements before and after processing indicate an uptake of 3.3% of reacted cyclodextrin based on the weight of the fabric.

Diagnostic test by discolouration of a basic purple phenolphthaleine solution reveals the presence of reacted cyclodextrin on cotton before and after machine wash at 50° C. with detergent ECE77.

### Example A5

Pad-Dry (120° C.) Thermofix (180° C.) on Bleached Mercerized Cretonne:

5.79 gram of a cotton fabric (bleached mercerized cretonne; 140 g/m²) is padded at room temperature (22° C.) with the bath obtained according to Example A1. The pick up ratio is 69.1%. After drying for 60 sec at 120° C. in a drying tenter, the fabric is thermofixed in the same apparatus for 60 sec at 180° C. Unreacted material, urea and salts are removed by rinsing 5 times with water at 20° C. for 10 min until the pH is stable at around 8 (bath ratio 1/90). The fabric is then air dried. Accurate mass measurements before and after processing indicate an uptake of 4.1% of reacted cyclodextrin based on the weight of the fabric.

Diagnostic test by discolouration of a basic purple phenolphthaleine solution reveals the presence of reacted cyclodextrin on cotton before and after machine wash at 50° C. with detergent ECE77.

### Example A6

Bath Recipe for Pad-Batch Cold Application:

43.6 milliliter of a bath are prepared containing 2.67 gram of the compound of Example 16, 8.5 milliliter of a sodium dicarbonate solution (100 g/l), 6.0 milliliter of a sodium hydroxide solution (1 N) and 29 milliliter of water having a pH of 12.7.

### Example A7

Pad-Batch Cold on Bleached Cretonne:

5.51 gram of a cotton fabric (bleached cretonne; 135 g/m²) is padded at room temperature (22° C.) with the bath obtained according to Example A6. The pick up ratio is 72.6%. The fabric is then rolled on a stainless steel rod which is kept in an air tight plastic cylinder for 17.5 hours in a bath thermostated at 25° C. The fabric is rinsed 5 times with water (bath ratio 1/90) for 10 min at 20° C. until the pH is stable at around 8-8.5 and air dried. Unreacted material and salts are removed by rinsing 5 times with water at 20° C. for 10 min until the pH is stable at around 8 (bath ratio 1/90). The fabric is then air dried. Accurate mass measurements before and after processing indicate an uptake of 4.2% of reacted cyclodextrin based on the weight of the fabric.

Diagnostic test by discolouration of a basic purple phenolphthaleine solution reveals the presence of reacted cyclodextrin on cotton before and after machine wash at 50° C. with detergent ECE77.

### Example A8

Pad-Batch Cold on Bleached Mercerized Cretonne:

5.22 gram of a cotton fabric (bleached mercerized cretone; 140 g/m²) is padded at room temperature (22° C.) with the bath obtained according to Example A6. The pick up ratio is 70.9%. The fabric is then rolled on a stainless steel rod which is kept in an air tight plastic cylinder for 17.5 hrs in a bath thermostated at 25° C. The fabric is rinsed 5 times with water (bath ratio 1/90) for 10 min at 20° C. until the pH is stable at around 8-8.5 and air dried. Unreacted material and salts are removed by rinsing 5 times with water at 20° C. for 10 min until the pH is stable at around 8 (bath ratio 1/90). The fabric is then air dried. Accurate mass measurements before and after processing indicate an uptake of 4.6% of reacted cyclodextrin based on the weight of the fabric.

Diagnostic test by discolouration of a basic purple phenolphthaleine solution reveals the presence of reacted cyclodextrin on cotton before and after machine wash at 50° C. with detergent ECE77.

### Comparative Examples A1 to A8

Experiments A1 to A8 are repeated in the absence of the reactive cyclodextrin derivative according to Example 16. In these cases no relevant mass increase of the fabric treated is detected.

### Example A9

Bath Recipe for Exhaust Application:

328 milliliter of a bath are prepared containing 1.47 grain of the compound of Example 16, 32.8 gram of sodium chloride and water.

### Example A10

Exhaust on Bleached Cretonne:

16.41 gram of a cotton fabric (bleached cretone; 135 g/m²) are clamped onto a fork which is immersed in 164 milliliter of the bath obtained according to Example A9 (liquor ratio 1/10) which was previously heated at 60° C. Efficient impregnation is provided by alternative vertical helicoïdal movement at a frequency of 60 per min for 30 min. 2.5 gram of anhydrous sodium carbonate are then added to the bath and the alternative movement is continued for 60 min at 60° C. The fabric is then removed from the bath and rinsed 5 times with water (bath ratio 1/90) for 10 min at 20° C. until the pH is stable at around 7.5-8. The residual bath after cooling has a pH of 10.5. The fabric is then air dried overnight. Accurate mass measurements before and after processing indicate an uptake of 1.5% of reacted cyclodextrin based on the weight of the fabric.

### Example A11

Exhaust on Bleached Mercerized Cretonne:

16.36 gram of a cotton fabric (bleached mercerized cretone; 140 g/m²) are clamped onto a fork which is immersed in 164 milliliter of the bath obtained according to Example A9 (liquor ratio 1/10) which was previously heated at 60° C. Efficient impregnation is provided by alternative vertical helicoïdal movement at a frequency of 60 per min for 30 min. 0.7 milliliter of aqueous sodium hydroxide (10 N) is then added to the bath and the alternative movement is continued for 60min at 60° C. The fabric is then removed from the bath and rinsed 5 times with water (bath ratio 1/90) for 10 min at 20° C. until the pH is stable around 7.5-8. The residual bath after cooling has a pH of 12.8. Accurate mass measurements before and after processing indicate an uptake of 1.0% of reacted cyclodextrin based on the weight of the fabric.

### Example A12

Concommittant Application of a Reactive Cyclodextrin and a Reactive Dye in the Same Padding Bath:

7.47 gram of cotton fabric (bleached creton, 135g/cm²) are padded at room temperature (22° C.) with a 51 milliliter bath containing 5.0 gram urea, 0.5 gram anhydrous sodium carbonate, 2.8 gram reactive compound obtained according to Example 16, 2.0 gram of Cibacron Blue C—R and water. The pick-up ratio is 73.6%. After drying for 60sec at 120° C. in a drying tenter, the fabric is thermofixed in the same apparatus for 60sec at 180° C. Unreacted materials, urea and salts are removed by rinsing 5 times with water at 20° C. for 10 min until pH is stable at around 8 (bath ratio 1/90), followed by boiling for 15 min. The fabric is then dried on a flat heating press for 30sec at 130° C.

Diagnostic test by discolouration of a basic purple phenolphthalein solution reveals the presence of reacted cyclodextrin on cotton before and after machine wash at 50° C.

1. A reactive polysaccharide derivative of formula 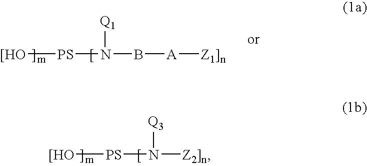
in which
A is —O—, —S— or 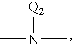
Q₁ is hydrogen, the radical —B—A—Z₁, C₁-C₁₀aryl which is unsubstituted or substituted, C₁-Cl₂alkyl which may be interrupted by oxygen and is unsubstituted or substituted,
Q₂ and Q₃ are each independently of the other hydrogen, C₁-C₁₀aryl which is unsubstituted or substituted, C₁-C₁₂alkyl which may be interrupted by oxygen and is unsubstituted or substituted,
B is an aliphatic or aromatic bridge member,
Z₁ and Z₂ are each independently of the other a reactive radical of the vinylsulfonyl series, the haloacryloyl series or the heterocyclic series,
PS is a polysaccharide radical,
m is 0, 1 or an integer greater than 1,
n is 1 or an integer greater than 1, and
the sum of n+m corresponds to the original number of hydroxy groups in the polysaccharide molecule.

2. A reactive polysaccharide derivative according to claim 1, wherein
Q₁ is hydrogen, benzyl and C₁-C₄alkyl which is unsubstituted or substituted by amino, or the radical —B—A—Z₁, and Q₂ and Q₃ are each independently of the other hydrogen, benzyl and C₁-C₄alkyl.

3. A reactive polysaccharide derivative according to claim 1, wherein
A is

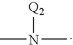

4. A reactive polysaccharide derivative according to claim 1, wherein
B is a C₂-C₁₂alkylene radical, which is unsubstituted or substituted by hydroxy, sulfo, sulfato, cyano or carboxy, and which may be interrupted by 1, 2 or 3 members from the group
—N(R₁ₐ)— and —O—, in which R₁ₐ is hydrogen or C₁-C₄alkyl, or R₁ₐ has the meaning indicated for Z₁ according to claim 1.

5. A reactive polysaccharide derivative according to claim 1, wherein
B is 1,2-ethylene, 1,3-propylene or 1,2-propylene.

6. A reactive polysaccharide derivative according to claim 1, wherein
Z₁ is a radical of formula (2a), (2b), (2c), (2d) or (2e) 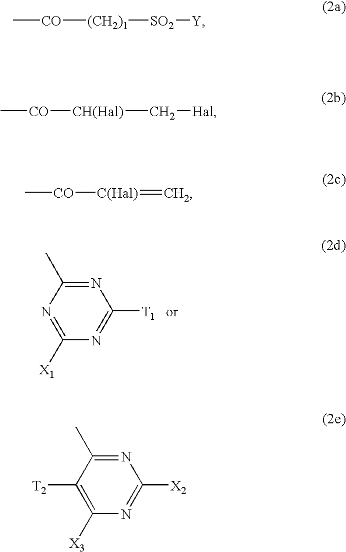
in which
Hal is chlorine or bromine,
X₁ is halogen, pyridinium, 3-carboxypyridin-1-yl or 3-carbamoylpyridin-1-yl, or a reactive radical of formula (3a), (3b), (3c), (3d), (3e) or (3f) 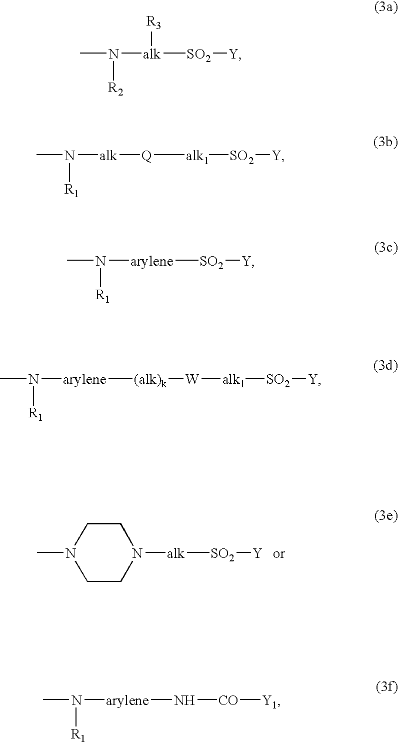
in which
R₁ is hydrogen or C₁-C₄alkyl,
R₂ is hydrogen, C₁-C₄alkyl unsubstituted or substituted by hydroxy, sulfo, sulfato, carboxy or by cyano, or a radical 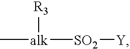
R₃ is hydrogen, hydroxy, sulfo, sulfato, carboxy, cyano, halogen, C₁-C₄alkoxycarbonyl, C₁-C₄alkanoyloxy, carbamoyl or a group —SO₂—Y,
alk and alk, are each independently of the other linear or branched C₁-C₆alkylene,
arylene is a phenylene or naphthylene radical unsubstituted or substituted by sulfo, carboxy, C₁-C₄alkyl, C₁-C₄alkoxy or by halogen,
Q is a radical —O— or —NR₁— wherein R₁ is as defined above,
W is a group —SO₂—NR₂—, —CONR₂— or —NR₂CO— wherein R₂ is as defined above,
Y is vinyl or a radical —CH₂—CH₂—U and U is a group removable under alkaline conditions,
Y₁ is a group —CH(Hal)—CH₂—Hal or —C(Hal)═CH₂ and Hal is chlorine or bromine, and
I is an integer from 1 to 6 and k is a number 0 or 1, and
X₂ is halogen or C₁-C₄alkylsulfonyl,
X₃ is halogen or C₁-C₄alkyl,
T₁ has independently the same definitions as X₁ above, or is a non-reactive substituent, and
T₂ is hydrogen, cyano or halogen.

7. A reactive polysaccharide derivative according to claim 1, wherein
Z₁ is a radical of formula (2a), (2b), (2c) or (2d) 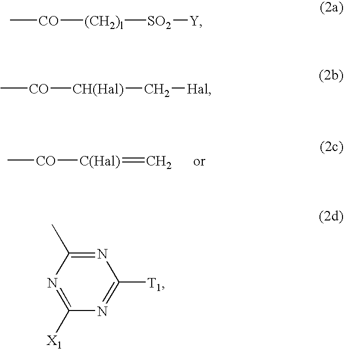
in which
Y is vinyl, β-chloroethyl or β-sulfatoethyl,
Hal is bromine, I is a number 2 or 3,
X₁ is halogen,
T₁ is C₁-C₄alkoxy, C₁-C₄alkylthio, hydroxy, amino, N-mono- or N,N-di-C₁-C₄alkylamino unsubstituted or substituted in the alkyl moiety by hydroxy, sulfato or by sulfo, morpholino, or phenylamino or N—C₁-C₄alkyl-N-phenylamino each unsubstituted or substituted in the phenyl ring by sulfo, carboxy, acetylamino, chlorine, methyl or by methoxy and wherein the alkyl is unsubstituted or substituted by hydroxy, sulfo or by sulfato, or naphthylamino unsubstituted or substituted by from 1 to 3 sulfo groups, or is a fibre-reactive radical of formula (3a′), (3b′), (3c′), (3d′) or (3f′) 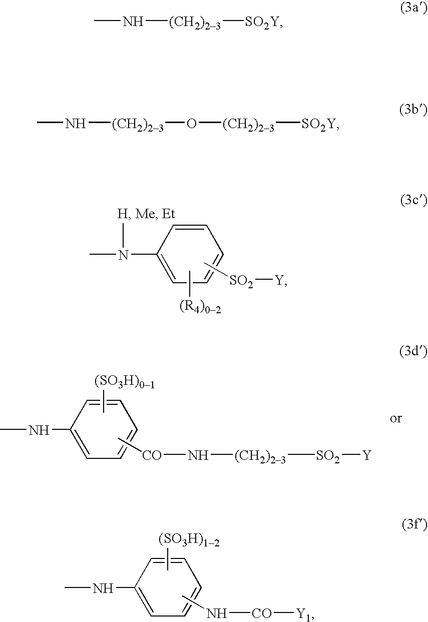
in which
(R₄)₀₋₂ is 0 to 2 identical or different substituents from the group of methyl, methoxy and sulfo,
Y is as defined above, and
Y₁ is a group —CH(Br)—CH₂—Br or —C(Br)═CH₂.

8. A reactive polysaccharide derivative according to claim 1, wherein
Z₂ is a radical of formula (4a), (4b), (4c), (4d), (4e) or (4f) 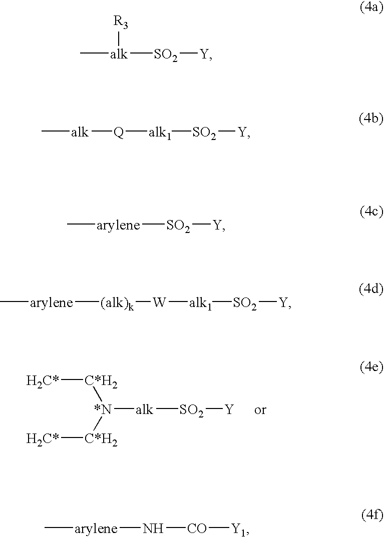
in which
R₃ is hydrogen, hydroxy, sulfo, sulfato, carboxy, cyano, halogen, C₁-C₄alkoxycarbonyl, C₁-C₄alkanoyloxy, carbamoyl or a group —SO₂—Y,
alk and alk, are each independently of the other linear or branched C₁-C₆alkylene,
arylene is a phenylene or naphthylene radical unsubstituted or substituted by sulfo, carboxy, C₁-C₄alkyl, C₁-C₄alkoxy or by halogen,
Q is a radical —O—,
W is a group —SO₂—NR₂—, —CONR₂— or —NR₂CO— wherein R₂ is hydrogen, C₁-C₄alkyl unsubstituted or substituted by hydroxy, sulfo, sulfato, carboxy or by cyano, or a radical 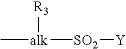
and R₃ is hydrogen, hydroxy, sulfo, sulfato, carboxy, cyano, halogen, C₁-C₄alkoxycarbonyl, C₁-C₄alkanoyloxy, carbamoyl or a group —SO₂—Y,
Y is vinyl or a radical —CH₂—CH₂—U and U is a group removable under alkaline conditions,
Y₁ is a group —CH(Hal)—CH₂—Hal or —C(Hal)═CH₂ and Hal is chlorine or bromine, and
k is a number 0 or 1, and
the atoms indicated with an asterisk in the reactive radical of formula (4e) together with the radical of formula 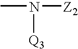
form a piperazine ring.

9. A reactive polysaccharide derivative according to claim 1, wherein
Z₂ is a radical of formula (4a′), (4b′), (4c′), (4c*), (4d′), (4d*) or (4f′) 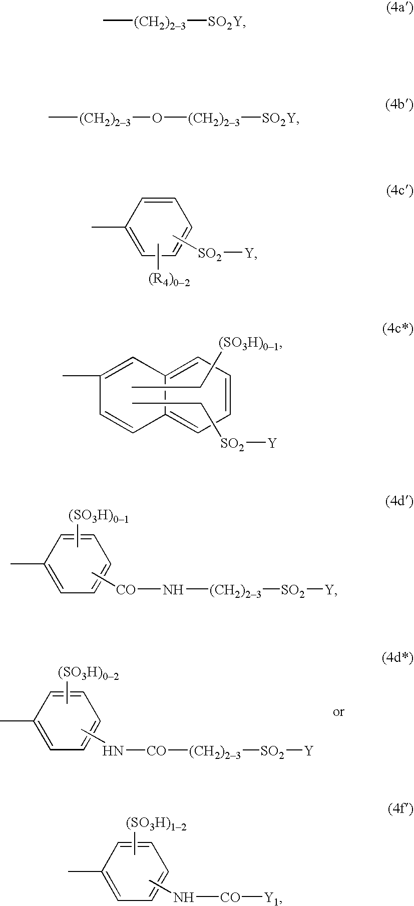
in which
(R₄)₀₋₂ is 0 to 2 identical or different substituents from the group of methyl, methoxy and sulfo,
Y is vinyl, β-chloroethyl or β-sulfatoethyl, and
Y₁ is a group —CH(Br)—CH₂—Br or —C(Br)═CH₂.

10. A reactive polysaccharide derivative according to claim 1, wherein
n is 1 or 2.

11. A process for the preparation of a reactive polysaccharide derivative of formula (1a) or (1b) according to claim 1, which process comprises the steps of
(i) introducing at least one leaving group into the polysaccharide molecule by reaction of a polysaccharide compound of the formula 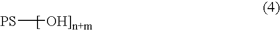
with at least n molar equivalents of a leaving group precursor P* to yield the compound of formula 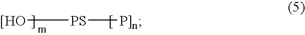
(ii) reacting the compound of formula (5) with at least n molar equivalents of the compound of the formula 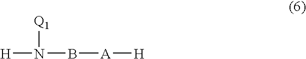
to yield the compound of formula 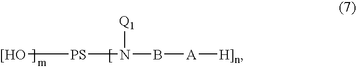
and allowing the compound of the formula (7) to react with at least n molar equivalents of the compound of the formula
Z₁—X (8),
or
reacting the compound of formula (5) with at least n molar equivalents of the compound of the formula 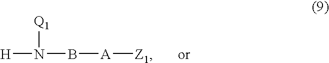
reacting the compound of formula (5) with at least n molar equivalents of the compound of the formula 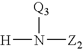
wherein
PS, Q₁, Q₃, A, B, Z₁, Z₂, m and n are as defined in claim 1, and X and P are each a leaving group.

12. A process according to claim 11, wherein
the compound of formula (4) corresponds to cyclodextrin or a cyclodextrin derivative.

13. A process for the preparation of compounds or substrates modified with polysaccharides comprising reacting the said compounds or substrates with a polysaccharide derivative according to claim 1.

14. A process for finishing textile fiber materials containing hydroxy groups or containing nitrogen, which comprises finishing said materials with a polysaccharide derivative according to claim 1.

15. A process according to claim 14, wherein the textile fiber materials are cellulose containing fiber materials.

16. A compound of formula 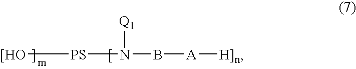
wherein PS, Q₁, A, B, m and n are as defined in claim 1,
with the exception of β-cyclodextrin which is substituted in the 6-position of one of the D-glucopyranosyl units by 2-aminoethylenamino or 2-hydroxyethylenamino and γ-cyclodextrin which is substituted in the 6-position of one of the D-glucopyranosyl units by 2-aminoethylenamino.
